(19) United States
(12) Patent Application Publication
(10) Pub. No.: US 2008/0015139 A1
(43) Pub. Date: Jan. 17, 2008

(54) Soluble virus-specific T-cell receptor compositions

(76) Inventors: Mathias Lichterfeld, Boston, MA (US); Xu Yu, Boston, MA (US); Bruce Walker, Nahant, MA (US); Marcus Altfeld, Arlington, MA (US); Georg Lauer, Somerville, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/784,277

(22) Filed: Apr. 5, 2007

Related U.S. Application Data
(60) Provisional application No. 60/789,790, filed on Apr. 5, 2006.

(51) Int. Cl. A61K 38/00; A61P 43/00; C07H 21/04; C07K 14/00; C12Q 1/70

(52) U.S. Cl. 514/2; 435/5; 530/326; 530/327; 530/328; 536/23.5

(57) ABSTRACT

Compositions and methods for diagnosing a viral infection and methods of inhibiting such an infection are described. The methods are based on the identification of T-cell receptor gene sequences from cytotoxic T cell clones that are specific for HIV-1 or HCV. Soluble T-cell receptor compositions that bind to HLA class I-restricted of HIV and HCV pathogens were identified and constructed.

### RELATED APPLICATIONS

This application claims priority to U.S. provisional patent application No. 60/789,790, filed on Apr. 5, 2006, the entire contents of which are hereby incorporated by reference.

### FIELD OF THE INVENTION

This invention relates to viral infection and diagnosis thereof.

### BACKGROUND OF THE INVENTION

Viral infections such as infection with human immunodeficiency virus (HIV) such as HIV-1 and infection with hepatitis C virus (HCV) are a significant public health problem. A number of drugs are currently used to treat individuals infected with such viral infections, e.g., anti-retroviral drugs include Reverse transcriptase (RT) inhibitors and Protease inhibitors (PI). Although these agents have been effective in treating HIV infection, development of drug resistant strains and cumulative drug toxicity of the virus is a concern.

### SUMMARY OF THE INVENTION

The invention features methods and compositions for diagnosis and treatment of viral infections. The methods are based on the identification of T-cell receptor gene sequences from cytotoxic T cell clones that are specific for HIV-1 or HCV. Accordingly, the invention includes isolated T cell receptor genes and polypeptides encoded by the genes, which encode soluble T cell receptor polypeptides or proteins that specifically bind to cytotoxic T cell epitopes in HIV-1 or HCV and polypeptides encoded by the genes.

The epitope comprises or consists of a short, 8 to 11-mer peptide that is associated with a class I major histocompatibility complex (MHC) antigen such as an HLA molecule on the surface of a cell. The epitope contains polypeptide sequence corresponding to or derived from a naturally-occurring HIV or HCV protein.

The T cell receptor genes encode for both the alpha chain of the T cell receptor and the beta chain of the T cell receptor. Both the TCR alpha chain and the TCR beta chain comprise or consist of a constant region (C region), a variable region (V region) and a complementary-determining 3 region (CDR3) region. The CDR3 regions mediate the interaction with the antigenic peptide/MHC class I complex and consist of a random sequence of 1-90 nucleotides that are generated by somatic recombination. These gene sequences are used to construct recombinant HIV-1 or HCV-specific soluble TCR receptor molecules, which are used for diagnostic in vitro use and therapeutic in vivo use. For instance, these soluble TCRs can be used as a staining reagent to detect HIV-1 or HCV cytotoxic T cell epitope presentation in patient-derived tissue or fluid samples in vitro assays. Optionally, the soluble T cell receptor polypeptide can be associated with a detectable marker such as a fluorescent molecule. The detectable marker is linked to or conjugated to the receptor polypeptide to facilitate diagnostic methods. Moreover, a plurality of soluble single chain HLA class I-restricted T cell receptor polypeptides are immobilized on a solid support such as a chip or plate. For example, the TCRs are configured in a microarray format for identification and detection of processed viral epitopes. For therapeutic purposes, a cytotoxic molecule or cytokine is linked to or associated with the TCR.

Preferred soluble TCR constructs include the following sequences that correspond to TCR α,β chain pairs: Vb sequence CASSQGVTLLN (SEQ ID NO:4) and Va5 sequence CAETY (SEQ ID NO:6). This soluble TCR has an epitope specificity of SEQ ID NO:5 (HIV-1 vpr) in the context of HLA class I molecule A2. Derivatives of the sequence of the TCR are also within the scope of the invention. Derivative TCRs are characterized by a higher binding affinity to the HLA class I restricted epitope shown in Table 1 below. For example, a derivative soluble TCR construct relative to the reference sequences SEQ ID NO:4 and 6 may include 1, 2, 3, 4 or more conservative or non-conservative amino acid substitutions and is characterized by a binding affinity for the epitope that is increased compared to a construct containing the original reference sequence. A preferred soluble TCR construct with an epitope binding specificity for HCV include sequences that correspond to TCR α,β chain pairs: Va sequence CAVNEYGQNFV (SEQ ID NO:27) and Vb sequence CAWSGGLNTEAF (SEQ ID NO:29). This soluble TCR construct has an epitope binding specificity of SEQ ID NO:28, an HCV peptide that is also presented in the context of HLA class I molecule A2. These and other TCR sequences as well as their binding specificities and HLA restriction are shown in Table 1.

By “substantially pure” is meant a nucleic acid, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis. An isolated fragment of a protein means a peptide having a portion of the sequence of the reference protein which is less than the entire sequence, and does not contain the naturally occurring flanking regions. An isolated polypeptide lacks one or more amino acids, which immediately flank the reference fragment in the naturally-occurring molecule.

Where a particular polypeptide or nucleic acid molecule is said to have a specific percent identity to a reference polypeptide or nucleic acid molecule of a defined length, the percent identity is relative to the reference polypeptide or nucleic acid molecule. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide which is 50% identical to the reference polypeptide over its entire length. The same rule applies for nucleic acid molecules.

For polypeptides, the length of the reference polypeptide sequence will generally be at least 5 amino acids in length. For example, the peptide is 5, 6, 7, 8, 9, 10, 11, 12 amino acids in length. In some cases, the peptide is larger, e.g., 15, 20, 25 amino acids or more in length. For example, the peptide of a specific sequence, e.g., epitope sequence, is flanked by other amino acids that differ from those amino acids which flank the sequence in a naturally-occurring protein. For nucleic acids, the length of the reference nucleic acid sequence will generally be at least 15, 20, or 25 nucleotides in length. However, larger constructs, e.g., those that are at least 50 nucleotides, preferably at least 60 nucleotides, more preferably at least 75 nucleotides, and most preferably 100 nucleotides or 300 nucleotides.

The invention also encompasses derivative peptides corresponding to TCR sequences or epitope sequences. In the case of polypeptide sequences which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. In peptides in which amino acids substitutions are made relative to the reference sequence, the binding affinity of the T-cell receptor to its HLA-restricted epitope is increased. Alternatively, constructs containing derivative sequences have great stability, e.g., a longer half-life in a physiologically acceptable solution such as culture media or a bodily fluid such as blood, plasma, or serum. For example, the binding affinity and/or stability of such derivative peptides is at least 5%, 10%, 25%, 50%, 75%, 90%, 100%, 2-fold, 5-fold, 10-fold, 20-fold, or more relative to that of the reference peptide sequence. Optionally, derivative peptide sequences include additional amino acids that flank the reference sequences on the amino-terminal and/or the carboxy-terminal end of the sequence. As is described above, such constructs, which contain non-naturally occurring flanking sequences are characterized as having increased epitope binding affinity and/or increased stability. Nucleic acid sequences that encode such derivative peptide sequences are encompassed by the invention.

An isolated or purified nucleic acid molecule is one that is separated from the 5′ and 3′ coding sequences or non-coding sequences with which it is immediately contiguous in the naturally occurring genome of an organism. Isolated nucleic acid molecules include nucleic acid molecules which are not naturally occurring, e.g., nucleic acid molecules created by recombinant DNA techniques. The nucleic acids identified herein include a sequence that are at least 85%, 90%, 95%, 98%, 99% identical to a reference sequence and degenerate variants of a reference nucleic acid sequence.

Other features and advantages of the invention will be apparent from the following description of the preferred embodiments thereof, and from the claims. The entire contents of references cited herein are hereby incorporated by reference.

### DETAILED DESCRIPTION OF THE INVENTION

HIV-1- or HCV-specific cells recognizing defined, MHC class I restricted cytotoxic T cell epitopes were isolated by limiting dilution cloning. Cloned cells were stained with MHC class I tetramers refolded with the respective epitopic peptide, and tetramer-binding cells were sorted using a FACS ARIA instrument. mRNA of sorted cells was extracted, reverse transcribed and cDNA of the TCR gene was amplified by nested PCR. PCR products were ligated into a cloning vector used to transform E. coli. After bacterial amplification, vector inserts were purified and sequenced according to standard procedures. The sequences of the following TCRs were identified.

TABLE 1
TCR sequences

| | | Restriciting |
|---|---|---|
| TCR (α(a), β(b), chain pairs) | Epitope specificity | HLA class I type |
| Vb27-CASSLGQGLANYGYT-J1.2 | FL8 (FLKEKGGL) | B8 |
| SEQ ID NO:1 | SEQ ID NO:2 | |
| Va3-CAVRDLTGNQFY-J49 | HIV-1 (nef) | |
| SEQ ID NO:3 | | |
| Vb14-CASSQGVTLLN-J2.1 | AL9 (AIIRILQQL) | A2 |
| SEQ ID NO:4 | SEQ ID NO:5 | |
| Va5-CAETY-J36 | HIV-1 (vpr) | |
| SEQ ID NO:6 | | |
| Vb19-CASSIDGASNQPQH-J1.5 | SL9 (SLYNTVATL) | A2 |
| SEQ ID NO:7 | SEQ ID NO:8 | |
| Va13.2-CAENSDAGGTSYGKLT-J52 | HIV-1 (gag) | |
| SEQ ID NO:9 | | |
| Vb15-CATSRGAGSNTGELF-J2.2 | FL8 (FLKEKGGL) | B8 |
| SEQ ID NO:10 | SEQ ID NO:2 | |
| Va12.2-CAVRGSGTYKYI-J40 | HIV-1 (nef) | |
| SEQ ID NO:11 | | |
| Va19-CALSGNHSGGATNKLI-J32 | TW10 | B57 |
| SEQ ID NO:12 | (TSTLQEQIGW) | |
| Vb4.3-CASSPWTGGGQPQH-J1.5 | SEQ ID NO:13 | |
| SEQ ID NO:14 | HIV-1 (gag) | |
| Va5-CAASGGYQKVTFGTGTKLQVIP | KF11 | B57 |
| SEQ ID NO:15 | (KAFSPEVIPMF) | |
| Vb19-CASTGTYGYT-J1.2 | SEQ ID NO:17 | |
| SEQ ID NO:16 | HIV-1 (gag) | |
| Va12.3-CAMSAQQAGTALI-J15 | SL9 (SEGATPQDL) | B60 |
| SEQ ID NO:18 | SEQ ID NO:19 | |
| Vb11.2-CASSLVIMSEQY-J2.7 | HIV-1 (gag) | |
| SEQ ID NO:20 | | |
| Va13.1-CAATSGYALN-J41 | EI8 (EIYKRWII) | B8 |
| SEQ ID NO:21 | SEQ ID NO:22 | |
| Vb9-CASSVQGEFREKLF-J1.4 | HIV-1 (gag) | |
| SEQ ID NO:23 | | |
| Va27-CAGRDYKLS-J20 | KL9 (KEKGGLEGL) | B60 |
| SEQ ID NO:24 | SEQ ID NO:25 | |
| Vb20.1-CSARGDNPNTEAF-J1.1 | HIV-1 (nef) | |
| SEQ ID NO:26 | | |
| Va8.1-CAVNEYGQNFV-J26 | AL9 (ALYDVVTKL) | A2 |
| SEQ ID NO:27 | SEQ ID NO:28 | |
| Vb30-CAWSGGLNTEAF-J1.1 | (HCV) | |
| SEQ ID NO:29 | | |
| Vb27-CASSVRTGELF-J2.2 | QK10 | A3 and A11 |
| SEQ ID NO:30 | (QVPLRPMTYK) | |
| Va29-CAASFTQNGLT-J45 | SEQ ID NO:31 | |
| SEQ ID NO:32 | HIV-1 (nef) | |
| Vb9-CASSERDSQYQETQY-J2.5 | QK10 | A3 and A11 |
| SEQ ID NO:33 | (QVPLRPMTYK) | |
| Va29-CAASFTQNGLT-J45 | SEQ ID NO:31 | |
| SEQ ID NO:34 | HIV-1 (nef) | |
| Vb14-CASSPVLYEQY-J2.7 | QK10 | A3 and A11 |
| SEQ ID NO:35 | (QVPLRPMTYK | |
| Va39-CAVVAQGGSEKLV-J57 | SEQ ID NO:31 | |
| SEQ ID NO:36 | HIV-1 (nef) | |
| Vb9-CASSARAFPEGNQPQH-J1.5 | QK10 | A3 and A11 |
| SEQ ID NO:37 | (QVPLRPMTYK) | |
| Va39-CAVVAQGGSEKLV-J57 | SEQ ID NO:31 | |
| SEQ ID NO:38 | HIV-1 (nef) | |
| Vb10.2-CASSETNRVMEAF-J1.1 | QK10 | A3 |
| SEQ ID NO:39 | (QVPLRPMTYK) | |
| Va8.6-CAVSDPGFKTI-J9 | SEQ ID NO:31 | |
| SEQ ID NO:40 | HIV-1 (nef) | |
| Vb24-CATSAGRQRDTGELF-J2.2 | QK10 | A3 |
| SEQ ID NO:41 | (QVPLRPMTYK) | |
| Va8.6-CAVSDPGFKTI-J9 | SEQ ID NO:31 | |
| SEQ ID NO:42 | HIV-1 (nef) | |
| Vb25.1-CASSNGYEQY-J2.7 | KV10 | A2 |
| SEQ ID NO:43 | (KLVALGINAV) | |
| Va38.2-CAYRSDNDMR-J43 | SEQ ID NO:45 | |
| SEQ ID NO:44 | (HCV) | |
| Vb24.1-CATSSQDGQVYEQY-J2.7 | GT9 (GPRLGVRAT) | B7 |
| SEQ ID NO:46 | SEQ ID NO:48 | |
| Va24-CASYKAAGNKLT-J17 | (HCV) | |
| SEQ ID NO:47 | | |
| Vb7.9-CASSSPKDPSNQPQH-J1.5 | KK10 | B27 |
| SEQ ID NO:82 | (KRWIILGLNK) | |
| Va5-CAEDPTSSSGYALN-J4 | SEQ ID NO:83 | |
| SEQ ID NO:84 | (HIV-1 gag) | |

TABLE 2
Nucleotide sequences encoding TCRs

| | |
|---|---|
| HCV-A2-KV10 (KLVALGINAV) | |
| Vb25.1-CASSNGYEQY-J2.7 | (SEQ ID NO:43) |
| NNNNNNNNGNNNNNNNTCGCCCTTNNCAGTGGTTCA | (SEQ ID NO:49) |
| ACGCAGAGTACGCGGGGGGGAGACATCCTCTCTAGC | |
| CCCAACTGTGCCATGACTATCAGGCTCCTCTGCTAC | |
| ATGGGCTTTTATTTTCTGGGGGCAGGCCTCATGGAA | |
| GCTGACATCTACCAGACCCCAAGATACCTTGTTATA | |
| GGGACAGGAAAGAAGATCACTCTGGAATGTTCTCAA | |
| ACCATGGGCCATGACAAAATGTACTGGTATCAACAA | |
| GATCCAGGAATGGAACTACACCTCATCCACTATTCC | |
| TATGGAGTTAATTCCACAGAGAAGGGAGATCTTTCC | |
| TCTGAGTCAACAGTCTCCAGAATAAGGACGGAGCAT | |
| TTTCCCCTGACCCTGGAGTCTGCCAGGCCCTCACAT | |
| ACCTCTCAGTACCTCTGTGCCAGCAGTAATGGATAC | |
| GAGCAGTACTTCGGGCCGGGCACCAGGCTCACGGTC | |
| ACAGAGGACCTGAAAAACGTGTTCCCACCCGAGGTC | |
| GCTGTGTTTGAGCCATCAGAAGCAGAGATCTCCCAC | |
| ACCAAGGGCGAATTCGTTTAAACCTGCAGGACTAGT | |
| CCCTTTAGTGAGGGTTAATTCTGAGCTTGGCGTAAT | |
| CATGGTCATAGNNNNTTTCCTNN | |
| Va38.2-CAYRSDNDMR-J43 | (SEQ ID NO:44) |
| NNNNNNNGGNNNNNNNANTCGCCCTTNNNAGTGGTA | (SEQ ID NO:50) |
| TCAACGCAGAGTACGCGGGGAGAAGAGGAGGCTTCT | |
| CACCCTGCAGCAGGGACCTGTGAGCATGGCATGCCC | |
| TGGCTTCCTGTGGGCACTTGTGATCTCCACCTGTCT | |
| TGAATTTAGCATGGCTCAGACAGTCACTCAGTCTCA | |
| ACCAGAGATGTCTGTGCAGGGGGCAGAGACCGTGAC | |
| CCTGAGCTGCACATATGACACCAGTGAGAGTGATTA | |
| TTATTTATTCTGGTACAAGCAGCCTCCCAGCAGGCA | |
| GATGATTCTCGTTATTCGCCAAGAAGCTTATAAGCA | |
| ACAGAATGCAACAGAGAATCGTTTCTCTGTGAACTT | |
| CCAGAAAGCAGCCAAATCCTTCAGTCTCAAGATCTC | |
| AGACTCACAGCTGGGGGATGCCGCGATGTATTTCTG | |
| TGCTTATAGGAGCGACAATGACATGCGCTTTGGAGC | |
| AGGGACCAGACTGACAGTAAAACCAAATATCCAGAA | |
| CCCTGACCCTGCCGTGTACCAGCTGAGAGACTCTAA | |
| ATCCAGTGACAAGTCTGTCTGCCTATTCACCGATTT | |
| TGATTCTCAAACAAATGTGTCACAAAGTAAGGATTC | |
| TGATGTGTATAAGGGCGAATTCGTTTAAACCTGCAG | |
| GACTAGTCCCTTTAGTGAGGGTTAATTCTGAGCTTG | |
| GCGTANTCATGGTCATAGNNNNNTTTCNNNN | |
| HCV-B7-GT9 (GPRLGVRAT) | (SEQ ID NO:48) |
| Vb24.1-CATSSQDGQVYEQY-J2.7 | (SEQ ID NO:46) |
| NNNNNNNNNNNNNNCNNNTCGCCCTTAAGCAGTGGT | (SEQ ID NO:51) |
| ATCAACGCAGAGTACGCGGGGAGAGCTGGAAACACC | |
| TCCATCCTGCCTCTTCATGCCATGGCCTCCCTGCTC | |
| TTCTTCTGTGGGGCCTTTTATCTCCTGGGAACAGGG | |
| TCCATGGATGCTGATGTTACCCAGACCCCAAGGAAT | |
| AGGATCACAAAGACAGGAAAGAGGATTATGCTGGAA | |
| TGTTCTCAGACTAAGGGTCATGATAGAATGTACTGG | |
| TATCGACAAGACCCAGGACTGGGCCTACGGTTGATC | |
| TATTACTCCTTTGATGTCAAAGATATAAACAAAGGA | |
| GAGATCTCTGATGGATACAGTGTCTCTCGACAGGCA | |
| CAGGCTAAATTCTCCCTGTCCCTAGAGTCTGCCATC | |
| CCCAACCAGACAGCTCTTTACTTCTGTGCCACCAGT | |
| TCCCAGGACGGGCAAGTCTACGAGCAGTACTTCGGG | |
| CCGGGCACCAGGCTCACGGTCACAGAGGACCTGAAA | |
| AACGTGTTCCCACCCGAGGTCGCTGTGTTTGAGCCA | |
| TCAGAAGCAGAGATCTCCCACACCAAGGGCGAATTC | |
| GTTTAAACCTGCAGGACTAGTCCCTTTAGTGAGGGT | |
| TAATTCTGAGCTTGGCGTAATCATGGTCATAGCTNN | |
| NTTNNNNGNN | |
| Va24-CASYKAAGNKLT-J17 | (SEQ ID NO:47) |
| NNNNNNNNNNNNNCNNCNAATTCGCCCTTANGCAGT | (SEQ ID NO:52) |
| GGTATCAACGCAGAGTACGCGGGGGTTTTTCTGCTG | |
| TGGGTACGTGAGCAGGAAACATGGAGAAGAATCCTT | |
| TGGCAGCCCCATTACTAATCCTCTGGTTTCATCTTG | |
| ACTGCGTGAGCAGCATACTGAACGTGGAACAAAGTC | |
| CTCAGTCACTGCATGTTCAGGAGGGAGACAGCACCA | |
| ATTTCACCTGCAGCTTCCCTTCCAGCAATTTTTATG | |
| CCTTACACTGGTACAGATGGGAAACTGCAAAAAGCC | |
| CCGAGGCCTTGTTTGTAATGACTTTAAATGGGGATG | |
| AAAAGAAGAAAGGACGAATAAGTGCCACTCTTAATA | |
| CCAAGGAGGGTTACAGCTATTTGTACATCAAAGGAT | |
| CCCAGCCTGAAGACTCAGCCACATACCTCTGTGCCT | |
| CCTACAAAGCTGCAGGCAACAAGCTAACTTTTGGAG | |
| GAGGAACCAGGGTGCTAGTTAAACCAAATATCCAGA | |
| ACCCTGACCCTGCCGTGTACCAGCTGAGAGACTCTA | |
| AATCCAGTGACAAGTCTGTCTGCCTATTCACCGATT | |
| TTGATTCTCAAACAAATGTGTCACAAAGTAAGGATT | |
| CTGATGTGTATAAGGGCGAATTCGTTTAAACCTGCA | |
| GGACTAGTCCCTTTAGTGAGGGTTAATTCTGAGCTT | |
| GGCGTAATCATGGTCNTANNNTNNTNNNNNN | |
| HCV A2-AL9 Va8.1-CAVNEYGQNFV-J26 | (SEQ ID NO:27) |
| NNNNNNNNNNGNNNNCNNNTCNCCCTTATACNCATC | (SEQ ID NO:53) |
| AGAATCCTTACTTTGTGACACATTTGTTTGAGAATC | |
| AAAATCGGTGACTAGGCAGACAGACTTGTCACTGGA | |
| TTTAGAGTCTCTCAGCTGGTACACGGCAGGGTCAGG | |
| GTTCTGGATATAGGGCAGCACGGACAATCTGGTTCC | |
| GGGACCAAAGACAAAATTCTGACCATACTCATTCAC | |
| GGCACAGAAGTACTCAGCTGTGTCACTCCACTGCAC | |
| AGAGGGTTTCCTCAGATTAAAGGAGAATTTACTCTT | |
| TATAAATTCAGCCTCAAAGCCCTTGATGCCTTTAAC | |
| CAGTGGATCCCCTGAAAAGTACTTGAGGAGAAGCTG | |
| AAGGTGTTGACCAGGGTACTGGACATACCAGAAGAG | |
| ATTAACAGTTCCACCGTAGGAATAGTTGCATCCCAA | |
| CTCCAGTGAGGCTGCTTCAGAGAGAATTACGTGGTG | |
| GTTATGCTGGCTCACAGACTGGGCTCTGGCATCTCT | |
| CAGGGCAAAAATCATCCCCAGCACTGGTATGAGCAA | |
| CAGGAGCATGGCTGAGCTGTGGAAACACTGCAAGCG | |
| TCTCTTTGGAAATTCTCCTCGGGGCCAGTAGGAAAG | |
| TGGCTGGAACCCAGGTCTTGAGAATAGCGAGCGTGA | |
| GGAAGGTTGGGCTAGGCAAGTCTCTTGTTTTGGTAA | |
| GAATCCCCGCGTACTCTGCGTTGATACCACTGCTTA | |
| ANGGCGAATTCGTTTAAACCTGCNNNACTAGTCCCT | |
| TTAGTGAGGGTTAATTCTGAGCTTGGNGTAATCATG | |
| GTCNNNNNNTGTTTTCCNGN | |
| HCV A2-AL9 Vb30-CAWSGGLNTEAF-J1.1 | (SEQ ID NO:29) |
| NNNNNNNNNNNNNNNTCGCCCTTGGTGTGGGANNNC | (SEQ ID NO:54) |
| TCTGCTTCTGATGGCTCAAACACAGCGACCTCGGGT | |
| GGGAACACCTTGTTCAGGTCCTCTACAACTGTGAGT | |
| CTGGTGCCTTGTCCAAAGAAAGCTTCAGTGTTCAGG | |
| CCTCCCGACCAGGCACAGAGATAGAAGCCAGAGTCA | |
| CTGAGAAGGAGCTTCTTAGAACTCAGGATGAACTGC | |
| CGGTCCTGGGGTCTGGAGGCTGAGAGATTCTGGGGC | |
| ACCTCAGAGCTGATCTGGCCAATACCAACGGAGTAG | |
| AAGAGCAGCTGGAGGCCCCTGCCTGCAGCCTGTCGG | |
| TACCAGTATAGGTTGGGGTTTGATGTTCCCTCCACA | |
| GTGCACTCCAGAGAGAGCGGGCTGCCCACAGGCTGC | |
| ACCAGGGTCGCTGGCCATTGATGAATAGTCTGAGAT | |
| CTGACCCCAAAGAAAGTGCCCAGGAGAAGGGCAAGG | |
| AGAGAGCAGAGCATCATCCAAGCCAGCCTTTCCTTC | |
| AGCTAGTCTGGGGGACAGACGCCTCCTCTTCTGGGC | |
| CAGTGATGTGGCCAGGCACACCAGTGTGGCCCGCGT | |
| ACTCTGCGTTGATACCACTGCTTAAGGGCGAATTCG | |
| CGGCCGCTAAATTCAATTCGCCCTATAGTGAGTCGT | |
| ATTACAATTCACTGGCNNNNCNNTTTTAN | |
| B8-E18 Va13.1-CAATSGYALN-J41 | (SEQ ID NO:21) |
| NNNNNNNNNNNNNCGCCCTTATACACATCAGAATCC | (SEQ ID NO:55) |
| TTACTTTGTGACACATTTGTTTGAGAATCAAAATCG | |
| GTGAATAGGCAGACAGACTTGTCACTGGATTTAGAG | |
| TCTCTCAGCTGGTACACGGCAGGGTCAGGGTTCTGG | |
| ATATGGGGTGTGACCAACAGCGAGGTGCCTTTGCCG | |
| AAGTTGAGTGCATACCCGGACGTTGCTGCACAGAAG | |
| TAGACAGCCGAGTCTTCAGGTTGGGTCTCTGTGATG | |
| TGCAGGGAGAAATGTTTGGCTGTCTTGTTCAATGTA | |
| ACAGCAATTCGTTGGTCTTTCTTTTCGCCCACATTT | |
| GAACGAATGTCTATAATAAGCTGAGGTCTTTTTCCA | |
| AGTTCTTGCTTATACCAAGGGAAGTAGTTTGAGGCA | |
| CTGTCTGAATAAGTACACTTGATAACAGCGCTGTCT | |
| CCCTCCTGGACACTCAGGGTTGAAGGATGCTGCTCC | |
| ACATTCTCTCCATTCACCAAGTCCAGCTGCAGCCAC | |
| AGGAATATAAATACAGCTCGAATGGATGTCATCCTT | |
| GTTCTTCCCAATTAAGATCAGTCATTGACCTGCAAC | |
| CTCCAGTTATCCCCGCGTACTCTGCGTTGATACCAC | |
| TGCTTAAGGGCGAATTCGCGGCCGCTAAATTCAATT | |
| CGCCCTATAGTGAGTCGTATTACAATTCACTGGCNT | |
| NCCNTTTTTAN | |
| B8-E18 Vb9-CASSVQGEFREKLF-J1.4 | (SEQ ID NO:23) |
| NNNNNNNNNNNNNNNNCGCCCTTGGTGTGGGANANC | (SEQ ID NO:56) |
| TCTGCTTCTGATGGCTCAAACACAGCGACCTCGGGT | |
| GGGAACACCTTGTTCAGGTCCTCCAAGACAGAGAGC | |
| TGGGTTCCACTGCCAAAAAACAGTTTTTCTCTAAAC | |
| TCCCCCTGTACGCTGCTGGCACAGAAATACAAAGCT | |
| GAGTCCCCCAGCTCCAGAGAGCTCAGGTTTAGTTCA | |
| GAGTGCAAGTCAGGGAACTGTTGTGCGGAGAATCGT | |
| TCAAGAATGTTTCCTTTTGCTCTCTCTTCTCCATTA | |
| TAATACTGAATGAGGAACTGGAGGCCCTGGTCCAGG | |
| CTCTGTTGGTACCAGTACACAGAGAGGTCTCCAGAC | |
| CTAGGGGAGCATCTCAGCGTCACTCGCTGTCCAGTT | |
| GCTGTGATCAGGTGCTTTGGGGTTTGTGTGACTCCA | |
| GAATCCACTGGGCCTGCTCCCAGGAGACAAAAGGCC | |
| ACACAGCAGAGGAGCCTGAAGCCCATGGCAGGATCT | |
| CCTAGCTTGGGGCTGGTGTCTCTGTAGTAAGCATTC | |
| TCCCCGCGTACTCTGCGTTGATACCACTGCTTAAGG | |
| GCGAATTCGCGGCCGCTAAATTCAATTCGCCCTATA | |
| GTGAGTCGTATTACAATTCACTGGCCNNNNNTTTTT | |
| ACANNNN | |
| B60-KL9 Va27-CAGRDYKLS-J20 | (SEQ ID NO:24) |
| NNNNNNNNNNNNNCGCCCTTATACACATCAGAATCC | (SEQ ID NO:57) |
| TTACTTTGTGACACATTTGTTTGAGAATCAAAATCG | |
| GTGAATAGGCAGACAGACTTGTCACTGGATTTAGAG | |
| TCTCTCAGCTGGTACACGGCAGGGTCAGGGTTCTGG | |
| ATATTTGCTCTTACAGTTACTGTGGTTCCGGCTCCA | |
| AAGCTGAGCTTGTAGTCGCGTCCTGCACAGAGGTAG | |
| AGGCCTGTATCACCAGGCTGGGCCGCAGTGATGTGG | |
| AGAGAACTGTCCTTTCTTGCATCACCAAACTGAAAG | |
| GTTAGTCTCTTCAGCTTCTTCACTTCTCCACCCGTA | |
| ACTACTGTCACCAGGAGGACAGGACCTTCCCCAGGC | |
| TCCTGTCTGTACCATTGTAAGCTGGAAAAAACACTT | |
| GAGGAGTTGCAGTACACAGTGAGATTTTCTCCCTCT | |
| TGGATGCTTAGAAACTGAGGGCTCTGCTCCAGCAGC | |
| TGGGTGCTCACCCATGCCAACTGAATCCAAAGAATG | |
| GACACGGAGAATTTCAGGACCATCTTGTCTTTCTAT | |
| CACATGGTGGACATGGCCCCTGACTTTAGCTGCTCC | |
| TGAAAGAGCCCGTCCTGGAACANACTTCTCTGNNCT | |
| ANAANANTGCTTGCTGCCACCCACTTTGAGTTCCAT | |
| ANAAAGCCCCCCGCGACTCTGCGTTGATACCACTGC | |
| TTNAGGGCGANNTCNCGNNCNNTAAATTCAATTCGC | |
| CCTATAGTGAGTCGTANTACAATTCACTGGCNNNNN | |
| NNTTTTANN | |
| B60-KL9 VB20.1-CSARGDNPNTEAF-J1.1 | (SEQ ID NO:26) |
| NNNNNNNGGNNNCNNANTCGCCCTTANGCAGTGGTA | (SEQ ID NO:58) |
| TCAACGCAGAGTACGCGGTAAGCAGTGGTATCAACG | |
| CAGAGTACGCGGGAGAGAAGGTGGTGTGAGGCCATC | |
| ACGGAAGATGCTGCTGCTTCTGCTGCTTCTGGGGCC | |
| AGGCTCCGGGCTTGGTGCTGTCGTCTCTCAACATCC | |
| GAGCTGGGTTATCTGTAAGAGTGGAACCTCTGTGAA | |
| GATCGAGTGCCGTTCCCTGGACTTTCAGGCCACAAC | |
| TATGTTTTGGTATCGTCAGTTCCCGAAACAGAGTCT | |
| CATGCTGATGGCAACTTCCAATGAGGGCTCCAAGGC | |
| CACATACGAGCAAGGCGTCGAGAAGGACAAGTTTCT | |
| CATCAACCATGCAAGCCTGACCTTGTCCACTCTGAC | |
| AGTGACCAGTGCCCATCCTGAAGACAGCAGCTTCTA | |
| CATCTGCAGTGCTAGAGGGGACAACCCGAACACTGA | |
| AGCTTTCTTTGGACAAGGCACCAGACTCACAGTTGT | |
| AGAGGACCTGAACAAGGTGTTCCCACCCGAGGTCGC | |
| TGTGTTTGAGCCATCAGAAGCAGAGATCTCCCACAC | |
| CAAGGGCGAATTCGTTTAAACCTGCAGGACTAGTCC | |
| CTTTAGTGAGGGTTAATTCTGAGCTTGGCGTAATCA | |
| TGGTCATANNNNNNTTTCCTNN | |
| A2-AL9 alpha chain: | |
| Va5-CAETY-J36 | (SEQ ID NO:6) |
| NNNNNNNNNNNNCGCCCTTATACACATCAGAATCCT | (SEQ ID NO:59) |
| TACTTTGTGACACATTTGTTTGAGAATCAAAATCGG | |
| TGAATAGGCAGACAGACTTGTCACTGGATTTAGAGT | |
| CTCTCAGCTGGTACACGGCAGGGTCAGGCTTCTGGA | |
| TATAGGGAATAACGGTGAGTCTCGTTCCAGTCCCAA | |
| AGAAGAGGTTGTTTGCCCCAGTTTGATAAGTCTCTG | |
| CACAGAAGTAGATAGCTGAGTCCCCAGTCTGGGTGT | |
| CTGCAATGCGCAGAGACAGATGTTTATCCTTTTTAT | |
| TCAATAGAACAGTGAGTCTTTGGTCTTGTTTCATGT | |
| CCATATTTGAGAAAATATACGTCAGCAACTGGAGAC | |
| CTGCTCCAGATTCTTGCTTATACCAGTATAAGTAGG | |
| TGGAGGAGCTGTCTGTGTAAGTGCAGTTTATAACGG | |
| AGCTGTCTCCCTCTCGGACACTCAGGAAAAGACTCT | |
| GCTCCACATCCTCTCCTCTACTCATACAGTCCAGCT | |
| GCAGCCACAAAAACAGGAACGAAAATCCAGCAAATG | |
| TCTTCATTGTTCTCCCCACTGGGACCTGCCCCGCGT | |
| ACTCTGCGTTGATACCACTGCTTAAGGGCGAATTCG | |
| CGGCCGCTAAATTCAATTCGCCCTATAGTGAGTCGT | |
| ATTACAATTCACTGNNNNNNNNTTTNNNNN | |
| A2-AL9 beta chain: | |
| Vb14-CASSQGVTLLN-J2.1 | (SEQ ID NO:4) |
| NNNNNNNNNNNNNNNCGCCCTTGGTGTGGGAGANCT | (SEQ ID NO:60) |
| CTGCTTCTGATGGCTCAAACACAGCGACCTCGGGTG | |
| GGAACACGTTTTTCAGGTCCTCTAGCACGGTGAGCC | |
| GTGTCCCTGGCCCGAAGAACTGCTCATTCAACAAAG | |
| TCACCCCTTGGCTGCTGGCACAGAAATAAACTCCAG | |
| AATCCTCCAGTTCTGCAGGCTGCACCTTCAGAGTAG | |
| AATACGTCCCTCCAGTCCTTTCAGCTAAGAATCGAT | |
| TGTTGGGCATACCGGACTCATCCTGTTTAGACTCTT | |
| TCACAAAATGTAACAGAAATTTTATTTCTTTTCCCA | |
| TAACACGTCGATACCAATAAAGATTATCATGTCCAG | |
| AAATTGGGTCACATCTCAGAGTCACAGTCTGGCCCT | |
| TCTCTATTACGCTGTGGCTGGGGAACTGAGTAACTC | |
| CAGCTTCTATGTGCTAAGCATGAGAAAAAGGAAAGC | |
| AAATCTGTCTCTTGGCCCTGTAAGATGTGGCCTCCA | |
| GTGACATCAGTATATTAGCCAATGTCCACAGTCTCA | |
| GGAGCTCCCTCTACCCCGCGTACTCTGCGTTGATAC | |
| CACTGCTTAAGGGCGAATTCGCGGCCGCTAAATTCA | |
| ATTCGCCCTATAGTGAGTCGTATTACAATTCACTGG | |
| CN | |
| B8-FL8 alpha chain: | |
| Va12.2-CAVRGSGTYKYI-J40 | (SEQ ID NO:11) |
| NNNNGNNCNNANTCGCCCTTNAGCAGTGGTATCAAC | (SEQ ID NO:61) |
| GCAGAGTACGCGGGGAAGAATGATGAAATCCTTGAG | |
| AGTTTTACTAGTGATCCTGTGGCTTCAGTTGAGCTG | |
| GGTTTGGAGCCAACAGAAGGAGGTGGAGCAGAATTC | |
| TGGACCCCTCAGTGTTCCAGAGGGAGCCATTGCCTC | |
| TCTCAACTGCACTTACAGTGACCGAGTTTCCCAGTC | |
| CTTCTTCTGGTACAGACAATATTCTGGGAAAAGCCC | |
| TGAGTTGATAATGTCCATATACTCCAATGGTGACAA | |
| AGAAGATGGAAGGTTTACAGCACAGCTCAATAAAGC | |
| CAGCCAGTATGTTTCTCTGCTCATCAGAGACTCCCA | |
| GCCCAGTGATTCAGCCACCTACCTCTGTGCCGTGCG | |
| AGGCTCAGGAACCTACAAATACATCTTTGGAACAGG | |
| CACCAGGCTGAAGGTTTTAGCAAATATCCNGAACCC | |
| TGACCCTGCCGTGTACCAGCTGAGAGACTCTAAATC | |
| CAGTGACAAGTCTGTCTGCCTATTCACCGATTTTGA | |
| TTCTCAAACAAATGTGTCACAAAGTAAGGATTCTGA | |
| TGTGTATAANGGCGAATTCGTTTAAACCTGCAGGAC | |
| TAGTCCCTTTAGTGAGGGTTAATTCTGANCTTGGCG | |
| TANTCATGGNNNNNNNNNNNNTTNNNNNNN | |
| B8-FL8 beta chain: | |
| Vb15-CATSRGAGSNTGELF-J2.2 | (SEQ ID NO:10) |
| NNNNNNNNCNNNNTCGCCCTTANGCAGTGGTATCAC | (SEQ ID NO:62) |
| GCAGAGTCGCGGGGGAGACAGACAGATGCTTCATTC | |
| CTGCATGGGGTGGTATTCCTGCCATGGGTCCTGGGC | |
| TTCTCCACTGGATGGCCCTTTGTCTCCTTGGAACAG | |
| GTCACGGGGATGCCATGGTCATCCAGAACCCAAGAT | |
| ACCAGGTTACCCAGTTTGGAAAGCCAGTGACCCTGA | |
| GTTGTTCTCAGACTTTGAACCATAACGTCATGTACT | |
| GGTACCAGCAGAAGTCAAGTCAGGCCCCAAAGCTGC | |
| TGTTCCACTACTATGACAAAGATTTTAACAATGAAG | |
| CAGACACCCCTGATAACTTCCAATCCAGGAGGCCGA | |
| ACACTTCTTTCTGCTTTCTTGACATCCGCTCACCAG | |
| GCCTGGGGGACGCAGCCATGTACCTGTGTGCCACCA | |
| GCAGAGGGGCAGGATCGAACACCGGGGAGCTGTTTT | |
| TTGGAGAAGGCTCTAGGCTGACCGTACTGGAGGACC | |
| TGAAAAACGTGTTCCCACCCGAGGTCGCTGTGTTTG | |
| AGCCATCAGAAGCAGAGATCTCCCACACCAAGGGCG | |
| AATTCGTTTAAACCTGCAGGACTAGTCCCTTTAGTG | |
| AGGGTTAATTCTGAGCTTGGCGTANTCATGGNNNNN | |
| NNNNTNTTTNCNNGN | |
| A2-SL9: Va13.2-CAENSDAGGTSYGKLT-J52 | (SEQ ID NO:9) |
| NNNNNNNGNNNCNNANTCGCCCTNNAGCAGTGGTAT | (SEQ ID NO:63) |
| CAACGCAGAGTACGCGGGGATGGCTGGAGATTGCAG | |
| GTTTATGACTGATCCTATTTGGGAAGAACAATGATG | |
| GCAGGCATTCGAGCTTTATTTATGTACTTGTGGCTG | |
| CAGCTGGACTGGGTGAGCAGAGGAGAGAGTGTGGGG | |
| CTGCATCTTCCTACCCTGAGTGTCCAGGAGGGTGAC | |
| AACTCTATTATCAACTGTGCTTATTCAAACAGCGCC | |
| TCANACTACTTCATTTGGTACAAGCAAGAATCTGGA | |
| AAAGATCCTCAATTCATTATAGACATTCGTTCAAAT | |
| ATGGACAAAAGGCAAGGCCAAAGAGTCACCGTTTTA | |
| TTGAATAAGACAGTGAAACATCTCTCTCTGCAAATT | |
| GCAGCTACTCAACCTGGAGACTCAGCTGTCTACTTT | |
| TGTGCAGAGAATTCTGATGCTGGTGGTACTAGCTAT | |
| GGAAAGCTGACATTTGGACAAGGGACCATCTTGACT | |
| GTCCATCCNAATATCCAGAAGCCTGACCCTGCCGTG | |
| TACCAGCTGAGAGACTCTAAATCCAGTGACAAGTCT | |
| GTCTGCCTATTCACCGATTTTGATTCTCAAACAAAT | |
| GTGTCACAAAGTAAGGATTCTGATGTGTATAAGGGC | |
| GAATTCGTTTAAACCTGCAGGACTAGTCCCTTTAGT | |
| GAGGGTTAATTCTGAGCTTGGCGTATCATGTNNNNN | |
| N | |
| A2-SL9: Vb19-CASSIDGASNQPQH-J1.5 | (SEQ ID NO:7) |
| NNNNNNNNGNNNNCNANTTCGCCCTTCCCTTTGCAC | (SEQ ID NO:64) |
| TATGAGCAACATTTGTTTCCTGGGAGCAAACACCGT | |
| GGATGGTGGAATCACTCAGTCCCCGAAGTACCTGTT | |
| CAGAAAGGAAGGACAGAATGTGACCCTGAGTTGTGA | |
| ACAGAATTTGAACCACGATGCCATGTACTGGTACCG | |
| ACAGGACCCAGGGCAAGGGCTGAGATTGATCTACTA | |
| CTCACAGATAGTAAATGACTTTCAGAAAGGAGGTAT | |
| AGCTGAAGGGTACAGCGTCTCTCGGGAGAAGAAGGA | |
| ATCCTTTCCTCTCACTGTGACATCGGCCCAAAAGAA | |
| CCCGACAGCTTTCTATCTCTGTGCCAGTAGTATAGA | |
| TGGCGCTAGCAATCAGCCCCAGCATTTTGGTGATGG | |
| GACTCGACTCTCCATCCTAGAGGACCTGAACAAGGT | |
| GTTCCCACCCGAGGTCGCTGTGTTTGAGCCATCAGA | |
| AGCAGAGATCTCCCACACCAAGGGCGAATTCGTTTA | |
| AACCTGCAGGACTAGTCCCTTTAGTGAGGGTTAATT | |
| CTGAGCTTGGCGTAATCATGGTCATANNNNNTTNNN | |
| NN | |
| B8-FL8: Vb27-CASSLGQGLANYGYT-J1.2 | (SEQ ID NO:1) |
| NNNNNNNNNNNNTNNNNNNGTCCTCTACNACGGTTA | (SEQ ID NO:65) |
| ACCTGGTCCCCGAACCGAAGGTGTAGCCATAGTTAG | |
| CTAAGCCCTGCCCTAAACTGCTGGCACAGAAGTACG | |
| GAGAGGTCTGGTTGGGGCTGGGCGACTCCAGGATCA | |
| GGGGGAAATTCCTCTTCTCTTTTCGAGAGACTTTGT | |
| ACCCTTCAGGAACACCTCCCTTATCAGTCACCTCAA | |
| CATTCATTGAATAGTAGATCTGCCTTAAGCCCAGCC | |
| CTGGGTCTTGTCGATACCAGGACATATACTCATGGT | |
| TCATATTCTGAGAACAAGTCACTGTTAACTTCTTTC | |
| CAGTCACTGTGATGAGGTATCTTGGGTTCTGGGTCA | |
| CTTGGGCTTCCAGGGGGCCTGCTCCTAGAAGGCAAA | |
| GGACCACATAGCCAAGGAGCTGGGGGCCCATGGCAG | |
| CATCAGGCAGGTGTCTGCCAGTTCTGGGGGCTCCAG | |
| GTGGTTTCTGTAACGTCTCCACCTCTTCCCCCGCGT | |
| ACTCTGCGTTGATACCACTGCNNNCNCTGCGTTGAN | |
| ACNNCTGNNN | |
| B8-FL8: Va3-CAVRDLTGNQFY-J49 | (SEQ ID NO:3) |
| GACCCCCCNNNNNNCGCCCGCCGNGAGCTTANNTGG | (SEQ ID NO:66) |
| AGCCATGGCCTCTGCACCCATCTCGATGCTTGCGAT | |
| GCTCTTCACATTGAGTGGGCTGAGAGCTCAGTCAGT | |
| GGCTCAGCCGGAAGATCAGGTCAACGTTGCTGAAGG | |
| GAATCCTCTGACTGTGAAATGCACCTATTCAGTCTC | |
| TGGAAACCCTTATCTTTTTTGGTATGTTCAATACCC | |
| CAACCGAGGCCTCCAGTTCCTTCTGAAATACATCAC | |
| AGGGGATAACCTGGTTAAAGGCAGCTATGGCTTTGA | |
| AGCTGAATTTAACAAGAGCCAAACCTCCTTCCACCT | |
| GAANAAACCATCTGCCCTTGTGAGCGACTCCGCTTT | |
| GTACTTCTGTGCTGTGAGAGACCTCACCGGTAACCA | |
| GTTCTATTTTGGGACAGGGACAAGTTTGACGGTCAT | |
| TCCAAATATCCAGAACCCTGACCCTGCCGTGTACCA | |
| GCTGANAGACTCTAAATCCAGTGACAAGTCTGTCTG | |
| CCTATTCACCGATTTTGATTCTCAAACAAATGTGTC | |
| ACAAANNNNN | |
| B57-TW10 Va19-CALSGNHSGGATNKLI-J32 | (SEQ ID NO:12) |
| NNNGGNCGCNNATTCGCCCTTAAGCAGTGGTATCAA | (SEQ ID NO:67) |
| CGCAGAGTACGCGGGGCAGTAACTTTGCTAGTACCT | |
| CTTGAGTGCAAGGTGGAGAATTAAGATCTGGATTTG | |
| AGACGGAGCACGGAACATTTCACTCAGGGGAAGAGC | |
| TATGAACATGCTGACTGCCAGCCTGTTGAGGGCAGT | |
| CATAGCCTCCATCTGTGTTGTATCCAGCATGGCTCA | |
| GAAGGTAACTCAAGCGCAGACTGAAATTTCTGTGGT | |
| GGAGAAGGAGGATGTGACCTTGGACTGTGTGTATGA | |
| AACCCGTGATACTACTTATTACTTATTCTGGTACAA | |
| GCAACCACCAAGTGGAGAATTGGTTTTCCTTATTCG | |
| TCGGAACTCTTTTGATGAGCAAAATGAAATAAGTGG | |
| TCGGTATTCTTGGAACTTCCAGAAATCCACCAGTTC | |
| CTTCAACTTCACCATCACAGCCTCACAAGTCGTGGA | |
| CTCAGCAGTATACTTCTGTGCTCTGAGTGGAAATCA | |
| CTCAGGTGGTGCTACAAACAAGCTCATCTTTGGAAC | |
| TGGCACTCTGCTTGCTGTCCGGCCAAATATCCAGAA | |
| CCCTGACCCTGCCGTGTACCAGCTGAGAGACTCTAA | |
| ATCCAGTGACAAGTCTGTCTGCCTATTCACCGATTT | |
| TGATTCTCAAACAAATGTGTCACAAAGTAAGGATTC | |
| TGATGTGTATAANGGCGAATTCGTTTAAACCTGCAN | |
| GGACTAGTCCCTTTAGTGAGGGNTAATTCTGANCTN | |
| GNCGNNATCNNNNNNNNNNNNNNNNNTNNNNNNNN | |
| B57-TW10 Vb4.3-CASSPWTGGGQPQH-J1.5 | (SEQ ID NO:14) |
| NNNNNNNGNNNNCNNNTTCGCCCTTANGCAGTGTAT | (SEQ ID NO:68) |
| CAACGCAGAGTACGCGGGAAGCAGTGGTATCAACGC | |
| AGAGTACGCGGGAAGCAGTGGTATCAACGCAGAGTA | |
| CGCGGGAAGCAGTGGTATCAACGCAGAGTACGCGGG | |
| AAGCAGTGGTATCAACGCAGAGTACGCGGGAAGCAG | |
| TGGTATCAACGCAGAGTACGCGGGGGTCATAACGCT | |
| ATGTATTGGTACAAGCAAAGTGCTAAGAAGCCACTG | |
| GAGCTCATGTTTGTCTACAGTCTTGAAGAACGGGTT | |
| GAAAACAACAGTGTGCCAAGTCGCTTCTCACCTGAA | |
| TGCCCCAACAGCTCTCACTTATTCCTTCACCTACAC | |
| ACCCTGCAGCCAGAAGACTCGGCCCTGTATCTCTGC | |
| GCCAGCAGCCCGTGGACAGGGGGCGGCCAGCCCCAG | |
| CATTTTGGTGATGGGACTCGACTCTCCATCCTAGAG | |
| GACCTGAACAAGGTGTTCCCACCCGAGGTCGCTGTG | |
| GTTGAGCCATCAGAAGCGAGATCTCCCACACCAAGG | |
| GCGAATTCGTTTAAACCTGCAGGACTAGTCCCTTTA | |
| GTGAGGGTTAATTCTGAGCTTGGCGTAATCATGGTC | |
| NTAGNNNNGTTTCCNGA | |
| B57-KF11 Va5-CAASGGYQKVTFGTGTKLQVIP | (SEQ ID NO:15) |
| NNNNNNNNNNNNNNTCNCCCTTNNNCNGNGGTNNCN | (SEQ ID NO:69) |
| NCGCNNAGNANNCGGGGGAAGANATACTTGNNNNTA | |
| TNGCTCTCTTGGCTGGAGATTGCAGGTCCCAGTGGG | |
| GAGAACAATGAAGACATTTGCTGGATTTTCGTTCCT | |
| GTTTTTGTGGCTGCAGCTGGACTGTATGAGTAGAGG | |
| AGAGGATGTGGAGCAGAGTCTTTTCCTGAGTGTCCG | |
| AGAGGGAGACAGCTCCGTTATAAACTGCACTTACAC | |
| AGACAGCTCCTCCACCTACTTATACTGGTATAAGCA | |
| AGAACCTGGAGCAGGTCTCCAGTTGCTGACGTATAT | |
| TTTTTCAAATATGGACATGAAACAAGACCAAAGACT | |
| CACTGTTCTATTGAATAAAAAGGATAAACATCTGTC | |
| TCTGCGCATTGCAGACACCCAGACTGGGGACTCAGC | |
| TATCTACTTCTGTGCAGCTTCTGGGGGTTACCAGAA | |
| AGTTACCTTTGGAACTGGAACAAAGCTCCAAGTCAT | |
| CCCAAATATCCAGAAGCCTGACCCTGCCGTGTACCA | |
| GCTGAGAGACTCTAAATCCAGTGACAAGTCTGTCTG | |
| CCTATTCACCGATTTTGATTCTCAAACAAATGTGTC | |
| ACAAAGTAAGGATTCTGATGTGTATATCACAGACAA | |
| AACTGTCCATAGACCTCATGTCTAGCACAGTTTTGT | |
| CTGTGATCCCGCGTACTCTGCGTTGATACCACTGCT | |
| TANNNGNCGAATTCGTTTAAACCTGCNNNACTAGTC | |
| CCTTTANTGAGGGTTAATTCTGANCTTGNNGTAATC | |
| NTGGNNNNNNCNNNNNNTTTNCCNGNNNNN | |
| B57-KF11 Vb19-CASTGTYGYT-J1.2 | (SEQ ID NO:16) |
| NNNNNNNNNCNCNNANTCGCCCTTAAGCAGTGGTAT | (SEQ ID NO:70) |
| CAACGCAGAGTACGCGGGGACATTAGGCCAGGAGAA | |
| GCCCCCGAGCCAAGTCTCTTTTCTCATTCTCTTCCA | |
| ACAAGTGCTTGGAGCTCCAAGAAGGCCCCCTTTGCA | |
| CTATGAGCAACCAGGTGCTCTGCTGTGTGGTCCTTT | |
| GTCTCCTGGGAGCAAACACCGTGGATGGTGGAATCA | |
| CTCAGTCCCCAAAGTACCTGTTCAGAAAGGAAGGAC | |
| AGAATGTGACCCTGAGTTGTGAACAGAATTTGAACC | |
| ACGATGCCATGTACTGGTACCGACAGGACCCAGGGC | |
| AAGGGCTGAGATCGATCTACTACTCACAGATAGTAA | |
| ATGACTTTCAGAAAGGAGATATAGCTGAAGGGTACA | |
| GCGTCTCTCGGGAGAAGAAGGAATCCTTTCCTCTCA | |
| CTGTGACATCGGCCCAAAAGAACCCGACAGCTTTCT | |
| ATCTCTGTGCCAGTACCGGGACTTATGGCTACACCT | |
| TCGGTTCGGGGACCAGGTTAACCGTTGTAGAGGACC | |
| TGAACAAGGTGTTCCCACCCGAGGTCGCTGTGTTTG | |
| AGCCATCAGAAGCAGAGATCTCCCACACCAAGGGCG | |
| AATTCGTTTAAACCTGCAGGACTAGTCCCTTTAGTG | |
| AGGGTTAATTCTGAGCTTGGCGTANTCATGGTCNNN | |
| NNNTNNNTTNCCNGNN | |
| B60-SL9 Va12.3-CAMSAQQAGTALI-J15 | (SEQ ID NO:18) |
| NNNNNNNGNNNNCNNNTCGCCCTTNAGCAGTGGTAT | (SEQ ID NO:71) |
| CAACGCAGAGTACGCGGGGAGGACAGATTTCTTTTA | |
| TGATTCCTACAGCAGAAAAATGAGAAACGTTTGTTA | |
| TTATTTTTTTTTCGTGTTTAAAGTTTGAATCCTCAG | |
| TGAACCAGGGCAGAAAAGAATGATGAAATCCTTGAG | |
| AGTTTTACTGGTGATCCTGTGGCTTCAGTTAAGCTG | |
| GGTTTGGAGCCAACAGAAGGAGGTGGAGCAGGATCC | |
| TGGACCACTCAGTGTTCCAGAGGGAGCCATTGTTTC | |
| TCTCAACTGCACTTACAGCAACAGTGCTTTTCAATA | |
| CTTCATGTGGTACAGACAGTATTCCAGAAAAGGCCC | |
| TGAGTTGCTGATGTACACATACTCCAGTGGTAACAA | |
| AGAAGATGGAAGGTTTACAGCACAGGTCGATAAATC | |
| CAGCAAGTATATCTCCTTGTTCATCAGAGACTCACA | |
| GCCCAGTGATTCAGCCACCTACCTCTGTGCAATGAG | |
| CGCGCAACAGGCAGGAACTGCTCTGATCTTTGGGAA | |
| GGGAACCACCTTATCAGTGAGTTCCAATATCCAGAA | |
| CCCTGACCCTGCCGTGTACCAGCTGAGAGACTCTAA | |
| ATCCAGTGACAAGTCTGTCTGCCTATTCACCGATTT | |
| TGANTCTCAAACAAATGTGTCACAAAGTAAGGATTC | |
| TGATGTGTATAANGGCGAATTCGTTTAAACCTGCAG | |
| GACTAGTCCCTTTAGTGAGGGTTAATTCTGAGCTTG | |
| GCGNNATCNNNNNNAANNNTNTTTTNNNNNNNNN | |
| B60-SL9 Vb11.2 | (SEQ ID NO:20) |
| Vb11.2-CASSLVIMSEQY-J2.7 | |
| NNNNNNNNGNNNCNNNTCGCCCTTGGTGTGGGAGAN | (SEQ ID NO:72) |
| CTCTGCTTCTGATGGCTCAAACACAGCGACCTCGGG | |
| TGGGAACACGTTTTTCAGGTCCTCTGTGACCGTGAG | |
| CCTGGTGCCCGGCCCGAAGTACTGCTCGCTCATGGT | |
| GACTAAGCTGCTGGCACAGAGATACACGGCCGAGTC | |
| CTCAAGCTTTGCAGGCTGGATCTTGAGAGTGGAGTC | |
| TACTCCTTTGAGCCTCTCTGCAGAAAATCGATCCTT | |
| AGGCAACTGTGAATCATCCACTACACCGTTATTCTG | |
| AAACTGAATCAGAAGCTTTGGGCCCTGTCCCAGGAT | |
| CTGCTGGTACCAGTAAAGGGTAGCATGGCCAGATAT | |
| AGGATTGCACCAAAAAGCCACACTCTGCCTTTTCTC | |
| TATAATCTTATATCTGGGAGACTGGGCAACTCCAGC | |
| TTCTGTGAGTTCTGCTCCCAGGAGACAGAGGGCCGC | |
| CCAGCAGAGGAGCCTGGTGCCCATGGCAGGGTCAGG | |
| GAAGGATGGGAGCTTTGCCCAATCAAGGTCACTGTG | |
| AGCAACAGCCCCCGCGTACTCTGCGTTGATACCACT | |
| GCTTAAGGGCGAATTCGTTTAAACCTGCAGGACTAG | |
| TCCCTTTAGTGAGGGTTAATTCTGAGCTTGGCGTNN | |
| TCATGGTNNNNNNNNNTTTNCCNNNN | |
| A3/A11 QK10 | |
| Vb27-CASSVRTGELF-J2.2 | (SEQ ID NO:30) |
| NNNNNNNNNNNANTCGCCCTTGGTGTGGGAGANCTC | (SEQ ID NO:73) |
| TGCTTCTGATGGCTCAAACACAGCGACCTCGGGTGG | |
| GAACACGTTTTTCAGGTCCTCCAGTACGGTCAGCCT | |
| AGAGCCTTCTCCAAAAAACAGCTCCCCGGTCCGTAC | |
| GCTGCTGGCACAGAAGTACAGAGAGGTCTGGTTGGG | |
| GCTCGGCGACTCCAGGATCAGGGGGAAATTCCTCTT | |
| CTCTTTTCGAGAGACTTTGTACCCTTCAGGAACATC | |
| TCCCTTATCAGTCACCTCAACATTCATTGAATAGTA | |
| GATCTGCCTTAAGCCCAGCCCTGGGTCTTGNTGNNA | |
| CCAGAACAGAATTCGAGAAGGGCGAATTCGCGGCCG | |
| CTAAATTCAATTCGCCCTATAGTGAGTCGTATTACA | |
| ATTCACTGGCCGNNNTTTTANNN | |
| Vb9-CASSERDSQYQETQY-J2.5 | (SEQ ID NO:33) |
| NNNNNNNNNNNNNTCGCCCTTGGTGTGGGAGANCTC | (SEQ ID NO:74) |
| TGCTTCTGATGGCTCAAACACAGCGACCTCGGGTGG | |
| GAACACGTTTTTCAGGTCCTCGAGCACCAGGAGCCG | |
| CGTGCCTGGCCCGAAGTACTGGGTCTCTTGGTACTG | |
| ACTGTCCCTCTCGCTGCTGGCACAGAAATACAAAGC | |
| TGAGTCCCCCAGCTCCAGAGAGCTCAGGTTTAGTTC | |
| AGAGTGCAAGTCAGGGAACTGTTGTGCGGAGAATCG | |
| TTCAAGAATGTTTCCTTTTGCTCTCTCTTCTCCATT | |
| ATAATACTGAATGAGGAACTGGAGGCCCTGGTCCAG | |
| GCTCTGATGGTACCANNACAGAATTCGAGAAGGGCG | |
| AATTCGCGGCCGCTAAATTCAATTCGCCCTATAGTG | |
| AGTCGTATTACAATTCACTGGCCGNNCGTTTTANAN | |
| Va29-CAASFTQNGLT-J45 | (SEQ ID NO:34) |
| NNNNNNNNNNNNANTCGCCCTTANCAGTGGTATCAA | (SEQ ID NO:75) |
| CGCAGAGTACGCGGGGGGACATGAATAAAGCACAGG | |
| AGGTTGAAGTCAGATTTGCAGCTTTCTAGGCGGGAG | |
| ACAAGACAATCTGCATCTTCACAGGGGGGATGGCCA | |
| TGCTCCTGGGGGCATCAGTGCTGATTCTGTGGCTTC | |
| AGCCAGACTGGGTAAACAGTCAACAGAAGAATGATG | |
| ACCAGCAAGTTAAGCAAAATTCACCATCCCTGAGCG | |
| TCCAGGAAGGAAGAATTTCTATTCTGAACTGTGACT | |
| ATACTAACAGCATGTTTGATTATTTCCTATGGTACA | |
| AAAAATACCCTGCTGAAGGTCCTACATTCCTGATAT | |
| CTATAAGTTCCATTGAGGATAAAAATGAAGATGGAA | |
| GATTCACTGTCTTCTTAAACAAAAGTGCCAAGCACC | |
| TCTCTCTGCACATTGTGCCCTCCCAGCCTGGAGACT | |
| CTGCAGTGTACTTCTGTGCAGCAAGCTTCACGCAGA | |
| ACGGACTCACCTTTGGCAAAGGGACTCATCTAATCA | |
| TCCAGCCCTATATCCAGAACCCTGACCCTGCCGTGT | |
| ACCAGCTGAGAGACTCTAACTCCAGTGACAAGTCTG | |
| TCTGCCTATTCACCGATTTTGATTCTCAAACAAATG | |
| TGTCACAAAGTAAGGATTCTGATGTGTATAANGNCG | |
| AATTCGCGGCCGCTAAATTCAATTCGCCCTATAGTG | |
| AGTCGTATTACAATTCACTGNNNNNCNNNNTTTNN | |
| A3/A11 QK10 | |
| Vb14-CASSPVLYEQY-J2.7 | (SEQ ID NO:35) |
| NNNNNNNNNNNNNCGCCCTTGGTGTGGGANANCTCT | (SEQ ID NO:76) |
| GCTTCTGATGGCTCAAACACAGCGACCTCGGGTGGG | |
| AACACGTTTTTCAGGTCCTCTGTGACCGTGAGCCTG | |
| GTGCCCGGCCCGAAGTACTGCTCGTATAGAACGGGG | |
| CTGCTGGCACAGAAATAAACTCCAGAATCCTCCAGT | |
| TCTGCAGGCTGCACCTTCAGAGTAGAATACGTCCCT | |
| CCAGTCCTTTCAGCTAAGAATCGATTGTTGGGCATA | |
| CCGGACTCATCCTGTTTAGACTCTTTCACAAAATGT | |
| AACAGAAATTTTATTTCTTTTCCCATAACATGTCGA | |
| TACCAGTACAGAATTCGAGAAGGGCGAATTCGCGGC | |
| CGCTAAATTCAATTCGCCCTATAGTGAGTCGTATTA | |
| CAATTCACTGGCCGNCGTTTTNNNN | |
| Vb9-CASSARAFPEGNQPQH-J1.5 | (SEQ ID NO:37) |
| NNNNNTNNNNNNNATTCGCCCTTGGTGTGGGANANC | (SEQ ID NO:77) |
| TCTGCTTCTGANGGCTCAAACACAGCGACCTCGGGT | |
| GGGAACACCTTGTTCAGGTCCTCTAGGATGGAGAGT | |
| CGAGTCCCATCACCAAAATGCTGGGGCTGATTGCCC | |
| TCTGGGAAGGCCCGGGCGCTGCTGGCACAGAAATAC | |
| AAAGCTGAGTCCCCCAGCTCCAGAGAGCTCAGGTTT | |
| AGTTCAGAGTGCAAGTCAGGGAACTGTTGTGCGGAG | |
| AATCGTTCAAGAATGTTTCCTTTTGCTCTCTCTTCT | |
| CCATTATAATAGTGAATGAGGAACTGGAGGCCCTGG | |
| TCCAGGCTCTGACGGTACCAGTACAGAATTCGAGAA | |
| GGGCGAATTCGCGGCCGCTAAATTCAATTCGCCCTA | |
| TAGTGAGTCGTATTACAATTCACTGGCCGTCGTTTT | |
| ANAN | |
| Va39-CAVVAQGGSEKLV-J57 | (SEQ ID NO:38) |
| NNNNNTNNNNNNNATTCGCCCTTGGTGTGGGANANC | (SEQ ID NO:78) |
| TCTGCTTCTGANGGCTCAAACACAGCGACCTCGGGT | |
| GGGAACACCTTGTTCAGGTCCTCTAGGATGGAGAGT | |
| CGAGTCCCATCACCAAAATGCTGGGGCTGATTGCCC | |
| TCTGGGAAGGCCCGGGCGCTGCTGGCACAGAAATAC | |
| AAAGCTGAGTCCCCCAGCTCCAGAGAGCTCAGGTTT | |
| AGTTCAGAGTGCAAGTCAGGGAACTGTTGTGCGGAG | |
| AATCGTTCAAGAATGTTTCCTTTTGCTCTCTCTTCT | |
| CCATTATAATAGTGAATGAGGAACTGGAGGCCCTGG | |
| TCCAGGCTCTGACGGTACCAGTACAGAATTCGAGAA | |
| GGGCGAATTCGCGGCCGCTAAATTCAATTCGCCCTA | |
| TAGTGAGTCGTATTACAATTCACTGGCCGTCGTTTT | |
| ANAN | |
| A3 QK10 Vb10.2-CASSETNRVMEAF-J1.1 | (SEQ ID NO:39) |
| NNNNNNNNANNNNTTCGCCCTTGGTGTGGGAGNNCT | (SEQ ID NO:79) |
| CTGCTTCTGATGGCTCAAACACAGCGACCTCGGGTG | |
| GGAACACCTTGTTCAGGTCCTCTACAACTGTGAGTC | |
| TGGTGCCTTGTCCAAAGAAAGCTTCCATTACCCTGT | |
| TTGTTTCACTGCTGGCGCAGAAATACACAGATGTCT | |
| GGGAGCGGGTAGCTGACTCCAGAGTGAGGGGGAAAT | |
| TCTCTGTCTTGGATCTGGAGACAACATAGCCATCGG | |
| GGACTTCTCCTTTATCTGTAATATCAGCAGCTGCTG | |
| AGTAATAGATCAGCCTCAGCCCATGTCCCAGGTCTT | |
| GACGGTACCAGAACAGAATTCGAGAAGGGCGAATTC | |
| GCGGCCGCTAAATTCAATTCGCCCTATAGTGAGTCG | |
| TATTACAATTCACTGGCCGTCGTTTTACN | |
| Vb24-CATSAGRQRDTGELF-J2.2 | (SEQ ID NO:41) |
| NNNNNNNNNNNNTCGCCCTTGGTGTGGGANNNCTCT | (SEQ ID NO:80) |
| GCTTCTGATGGCTCAAACACAGCGACCTCGGGTGGG | |
| AACACGTTTTTCAGGTCCTCCAGTACGGTCAGCCTA | |
| GAGCCTTCTCCAAAAAACAGCTCCCCGGTGTCTCGC | |
| TGCCTCCCGGCACTGGTGGCACAGAAGTAAAGAGCT | |
| GTCTGGTTGGGGATGGCAGACTCTAGGGACAGGGAG | |
| AATTTAGCCTGTGCCTGTCGAGAGACACTGTATCCA | |
| TCAGAGATCTCTCCTTTGTTTATATCTTTGACATCA | |
| AAGGAGTAATAGATCAACTGTAGGCCCAGTCCTGGG | |
| TCTTGATGGTACCAATACAGAATTCGAGAAGGGCGA | |
| ATTCGCGGCCGCTAAATTCAATTCGCCCTATAGTGA | |
| GTCGTATTACAATTCACTGGCCGTCGTTTTANNN | |
| Va8.6-CAVSDPGFKTI-J9 | (SEQ ID NO:40) |
| NNNNNNNNNNNNNANNNTCGCCCTTATACNCATCAG | (SEQ ID NO:81) |
| AATCCTTACTTTGTGACACATTTGTTTGAGAATCAA | |
| AATCGGTGAATAGGCAGACAGACTTGTCACTGGATT | |
| TAGAGTCTCTCAGCTGGTACACGGCAGGGTCAGGGT | |
| TCTGGATATTTGCTTTAACAAATAGTCTTGTTCCTG | |
| CTCCAAAGATAGTTTTGAAGCCTGGATCACTCACAG | |
| CACAGAAGTACTCAGCCGTGTCGCTTATATGGACTG | |
| AGGGTTTCCTCAAGTGGAAGGAAGTTTGACTCTTGT | |
| TAAATTCAGCCTCAAAACCGTTGATGCCTTTAACCA | |
| GGGTGGATCCTGATAAATACTTCAGGAGAAGCTGGA | |
| GTCCTTGGTTGGGGTATTGCACATACCAGAAGAGAT | |
| ACACTGAAACAGACGATGAGTAGTTGCACCTCAGCA | |
| CCACAGGGGCTTCTTCAAAGACAGGGACTTGGCTGT | |
| CAAGCTGGGTCACAGGCTGGGCTCTGGTTCCTCCCC | |
| GGGTAAAAATCACCTGGAACGCTGGGACGAGCAGCA | |
| GGAGCATGGCTGAGCAGTGGCAATGCTGCAGGACCT | |
| TGAGCTGGGCGGACAGAAGCCAAGGGCGCTGAGCCT | |
| CAGGAGCTAGGAACTGTGAGGAGGTTGGATTGGACA | |
| AGTCCCTGGCTTTGAAAAGTTTCAGAAACAGCCCCG | |
| CGTACTCCCCGCGTACTCTGCGTTGATACCACTGCT | |
| TAAGGGCGAATTCGCGGCCGCTAAATTCAATTCGCC | |
| CTATAGTGAGTCNNATTACAATTCACTGGCNN | |
| B27-KK10 Va5-CAEDPTSSSGYALN-J4 | (SEQ ID NO:84) |
| NNNNNNNNGGNNNCNNNTCGCCCTTAAGCAGTGGTA | (SEQ ID NO:85) |
| TCAACGCAGAGTACGCGGGGCAGGTCCCAGTGGGGA | |
| GAACAATGAAGACATTTGCTGGATTTTCGTTCCTGT | |
| TTTTGTGGCTGCAGCTGGACTGTATGAGTAGAGGAG | |
| AGGATGTGGAGCAGAGTCTTTTCCTGAGTGTCCGAG | |
| AGGGAGACAGCTCCGTTATAAACTGCACTTACACAG | |
| ACAGCTCCTCCACCTACTTATACTGGTATAAGCAAG | |
| AACCTGGAGCAGGTCTCCAGTTGCTGACGTATATTT | |
| TTTCAAATATGGACATGGAACAAGACCAAAGACTCA | |
| CTGTTCTATTGAATAAAAAGGATAAACATCTGTCTC | |
| TGCGCATTGCAGACACCCAGACTGGGGACTCAGCTA | |
| TCTACTTCTGTGCAGAGGATCCCACCTCAAGTTCCG | |
| GGTATGCACCCAACTTCGGCAAAGGCACCTCGCTGT | |
| TGGTCACACCCCATATCCAGAACCCTGACCCTGCCG | |
| TGTACCAGCTGAGAGACTCTAAATCCAGTGACAAGT | |
| CTGTCTGCCTATTCACCGATTTTGATTCTCAAACAA | |
| ATGTGTCACAAAGTAAGGATTCTGATGTGTATAAGG | |
| GCGAATTCGTTTAAACCTGCAGGACTAGTCCCTTTA | |
| GTGAGGGTTAATTCTGAGCTTGGCGTAATCNTNNNN | |
| NNNNNNNTTTTNNNNNNN | |
| Vb7.9-CASSSPKDPSNQPQH-J1.5 | (SEQ ID NO:82) |
| NNNNNNNNNNNNGCNNNTCGCCCTTNAGCAGTGGTA | (SEQ ID NO:86) |
| TCAACGCAGAGTACGCGGGGGATCTGGTAAAGCTCC | |
| CATCCTGCCCTGACCCTGCCATGGGCACCAGCCTCC | |
| TCTGCTGGATGGCCCTGTGTCTCCTGGGGGCAGATC | |
| ACGCAGATACTGGAGTCTCCCAGAACCCCAGACACA | |
| AGATCACAAAGAGGGGACAGAATGTAACTTTCAGGT | |
| GTGATCCAATTTCTGAACACAACCGCCTTTATTGGT | |
| ACCGACAGACCCTGGGGCAGGGCCCAGAGTTTCTGA | |
| CTTACTTCCAGAATGAAGCTCAACTAGAAAAATCAA | |
| GGCTGCTCAGTGATCGGTTCTCTGCAGAGAGGCCTA | |
| AGGGATCTTTCTCCACCTTGGAGATCCAGCGCACAG | |
| AGCAGGGGGACTCGGCCATGTATCTCTGTGCCAGCA | |
| GCAGTCCCAAAGATCCTAGCAATCAGCCCCAGCATT | |
| TTGGTGATGGGACTCGACTCTCCATCCTAGAGGACC | |
| TGAACAAGGTGTTCCCACCCGAGGTCGCTGTGTTTG | |
| AGCCATCAGAAGCAGAGATCTCCCACACCAAGGGCG | |
| AATTCGTTTAAACCTGCAGGACTAGTCCCTTTAGTG | |
| AGGGTTAATTCTGAGCTTGGCGTAATCNNNNNNNNN | |
| NNNTTTTTNNNNNNNN | |

Pathogen-Specific Soluble TCR Constructs

Molecular compounds that specifically recognize HIV-1 cytotoxic T cell epitopes bound to MHC class I molecules on the surface of HIV-1 infected cells are powerful tools for the direct targeting of infected cells for in vivo immunotherapeutic approaches. Moreover, these compounds are used for the diagnostic ex vivo assessment of HIV-1 antigen presented on lymphocytes or professional antigen presenting cells during natural infection. Soluble, single chain α/β T cell receptor constructs that specifically bind to cognate MHC complexes represent the most promising molecules for the direct ex vivo or in vivo targeting of HIV-1 infected cells.

The amino acid sequences of soluble TCRs recognizing a specific pathogen is based on the sequences of naturally-occurring TCRs. Prior to this disclosure, only very limited information was available on the TCR sequences of naturally occurring TCRs specific for HIV-1 or HCV epitopes. The data described herein elucidates sequences for HIV-1 or HCV-specific TCR genes that are used for the construction of soluble TCRs for diagnostic and therapeutic use.

Presently, recombinant HIV-1-specific antibodies are available for the direct targeting of HIV-1 infected cells. One drawback of the antibody approach is that only the envelope of the HIV-1 virus is accessible for HIV-1 antibodies, while the functionally most important HIV proteins are hidden inside the envelope and only accessible to the immune system after intracellular processing and presentation by MHC class I or II molecules. Once presented by MHC molecules, these HIV gene products are recognized by TCRs, but not by antibodies. HIV-1 antibodies therefore only allow for a very limited targeting of HIV-1 infected cells. The compositions described herein provide a solution to this problem.

The Soluble TCRs, which are Specific for HIV1 or HCV have Significant Advantages Over Existing Approaches

The complete sequences of TCR alpha and beta chains of naturally-occurring HIV-1-specific CD8+ T cell clones have been identified. These TCR sequences of HIV-1 or HCV-specific CD8+ T cells have been identified to date.

The TCR sequences are useful for the production of recombinant single chain TCR that are able to specifically recognize HIV-1 infected cells. These recombinant TCR are practically used for (i) the in vivo targeting of HIV-1 infected cells in immunotherapeutic approaches, (ii) the ex vivo assessment of HIV-1 antigen expression on lymphocytes or professional antigen presenting cells. The quantitative analysis of HIV-1 antigen expression is important in studies on HIV-1 immunopathogenesis and are useful for the ex vivo monitoring of immunotherapeutic treatment approaches.

Currently, treatment of HIV-1 infected patients is based on the use of antiretroviral drugs. These drugs are very effective, but have cumulative toxicity, are associated with high pill burdens and can lead to viral resistance. Therefore, there is a continuing need for other treatment options for these patients. Immunotherapeutic treatment approaches with soluble TCRs represent an alternative treatment option for the HIV-1 or HCV infected patient population. In addition, the TCR are used for the ex vivo assessment of HIV-1 antigen expression.

Methods of Diagnosis

Soluble TCRs are used to analyze HLA class I-mediated presentation of cytotoxic T cell epitope presentation on professional antigen presenting cells. For example, a sample of bodily fluid, e.g., blood, or bodily tissue, e.g., lymph node, is obtained from a subject. Leukocytes from the sample are contacted with single chain TCRs described herein. To increase sensitivity, four single chain TCR constructs linked together, e.g., with a central streptavidin to form a tetrameric complex. The construct is linked to a detectable marker, e.g., it is labeled with a fluorescence fluorophore. Detectable markers include fluorochromes such as Phycoerythrin (PE), Fluorescein isothiocyanate (FITC), and Allophycocyanin (APC). Detection is carried out by flow cytometry and/or tissue staining (immunohistochemistry). In another example, a plurality of TCR constructs are immobilized in a microarray, e.g., a chip or plate, and a patient-derived sample is allowed to contact the array, the array is washed, and bound cells detected. In this manner, the peptide expressed or presented on the antigen presenting cell of a patient is determined. Thus, soluble TCRs are also useful as a research tool for the ex vivo assessment and quantification of HIV-1 or HCV CTL epitope presentation. They are useful tools for identifying patients who express specific HIV-1 or HCV CTL epitopes, and are therefore promising candidates for immunotherapeutic interventions described herein.

Methods of Therapy

To treat patients infected with HCV or HIV, one or a mixture of soluble single chain TCR constructs are administered. The TCRs are conjugated to a second composition such as a cytokine, such as interleukin-2, interferon-gamma, interferon-alpha or cytotoxic reagents, such as perforin, granzyme or specific drugs. For treatment of HCV, the soluble single chain HCV-specific TCR is optionally conjugated or linked to an interferon such as interferon-alpha. One advantage of such a construct is increased half-life and the antigen-specific delivery of these reagents directly to infected cells. This therapeutic strategy reduces the overall drug dose, the dosing frequency, and the treatment-associated side effects.

A TCR construct is selected based on the genetic characteristics (e.g., prevalence of particular HLA type) of the target population. For example, a pool of soluble TCRs are used that recognize a repertoire of cytotoxic T cell epitopes that a restricted by the most frequently-occurring HLA class I molecules in a specific population. Alternatively, the HLA type of one particular patient is determined and one or more HLA specific TCRs are selected for administration based on the HLA type of the patient.

Parenteral administration, such as intravenous, subcutaneous, intramuscular, and intraperitoneal delivery routes, may be used to deliver soluble TCR constructs. For instance, soluble TCR have been intravenously injected into mice at a dose of 32 μg per animal. Determination of patient doses is carried using methods wells known in the art.

The compositions are administered to inhibit a viral pathogen. Determination of the proper dosage and administration regime for a particular situation is within the skill of the art. An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other agents or therapeutic agents. A therapeutic regimen is carried out by identifying a mammal, e.g., a human patient suffering from (or at risk of developing) infection by a viral pathogen, using standard methods. The pharmaceutical compound is administered to such an individual using methods known in the art. Preferably, the compound is administered orally, rectally, nasally, topically or parenterally, e.g., subcutaneously, intraperitoneally, intrathecally, intramuscularly, and intravenously.

### OTHER EMBODIMENTS

While the invention has been described in: conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising an isolated nucleic acid encoding a soluble HLA class I-restricted T cell receptor (TCR) polypeptide, which polypeptide specifically binds to an HIV or HCV epitope.

2. The composition of claim 1, wherein said nucleic acid encode an isolated TCR polypeptide comprising SEQ ID NO:4.

3. The composition of claim 1, wherein said nucleic acid encode an isolated TCR polypeptide comprising SEQ ID NO:6.

4. The composition of claim 1, wherein said nucleic acid encode an isolated TCR polypeptide comprising SEQ ID NO:4 and SEQ ID NO:6

5. The composition of claim 1, wherein said nucleic acid encode an isolated TCR polypeptide comprising SEQ ID NO:27.

6. The composition of claim 1, wherein said nucleic acid encode an isolated TCR polypeptide comprising SEQ ID NO:29.

7. The composition of claim 1, wherein said nucleic acid encode an isolated TCR polypeptide comprising SEQ ID NO:27 and SEQ ID NO: 29.

8. A composition comprising an isolated TCR polypeptide, which binds specifically to a HIV or HCV epitope.

9. The composition of claim 8, wherein said isolated TCR polypeptide comprises SEQ ID NO:4.

10. The composition of claim 8, wherein said isolated TCR polypeptide comprises SEQ ID NO:6.

11. The composition of claim 8, wherein said isolated TCR polypeptide comprises SEQ ID NO:4 and SEQ ID NO:6

12. The composition of claim 8, wherein said isolated TCR polypeptide comprises SEQ ID NO:27.

13. The composition of claim 8, wherein said isolated TCR polypeptide comprises SEQ ID NO:29.

14. The composition of claim 8, wherein said isolated TCR polypeptide comprises SEQ ID NO:27 and SEQ ID NO: 29.

15. The composition of claim 1 or 8, wherein said TCR polypeptide binds to an HIV-1 epitope.

16. The composition of claim 1 or 8, wherein said TCR polypeptide binds to an HCV epitope.

17. The nucleic acid of claim 1, wherein said nucleic acid comprises an alpha chain T cell receptor sequence and a beta chain receptor sequence.

18. The composition of claim 8, wherein said TCR polypeptide further comprises a detectable marker.

19. The TCR polypeptide of claim 18, wherein said detectable marker is a fluorochrome.

20. The TCR polypeptide of claim 8, wherein said TCR polypeptide further comprises a cytotoxic composition.

21. The TCR polypeptide of claim 8, wherein said TCR polypeptide further comprises a cytokine.

22. A composition comprising a plurality of soluble single chain HLA class I-restricted T cell receptor polypeptides of claim 8 immobilized on a solid support, wherein each of said plurality bind to different viral epitope

23. The composition of claim 8, wherein said TCR polypeptide comprises an alpha chain sequence and a beta chain sequence, each of said alpha and beta chain sequences being at least 8 residues in length.

24. The composition of claim 8, wherein each of said alpha and beta chain sequences are between 8 and 20 residues in length.

25. The composition of claim 8, wherein said polypeptide comprises an α chain sequence and a β chain sequence pair in Table 1.

26. The composition of claim 8, wherein said polypeptide comprises an alpha chain sequence selected from those listed in Table 1.

27. The composition of claim 8, wherein said polypeptide comprises a beta chain sequence selected from those listed in Table 1.

28. The composition of claim 1, wherein said nucleic acid comprises an alpha-chain encoding sequence selected from those listed in Table 2.

29. The composition of claim 2, wherein said nucleic acid comprises a beta chain encoding sequence is selected from those listed in Table 2.

30. A method of diagnosing a viral infection, comprising contacting an isolated virus-specific soluble T-cell receptor construct with a sample of a bodily fluid or tissue from a test subject and detecting binding to a T-cell receptor construct, wherein said binding indicates a viral infection.

31. A method of inhibiting a viral infection comprising administering to a subject an isolated single chain soluble virus specific T cell receptor, said receptor comprising a cytotoxic agent.

32. The method of claim 31, wherein said receptor specifically binds to an HIV or HCV epitope.


SEQUENCE LISTING (86 sequences)

SEQ ID NO: 1 - protein, 15 aa, Homo sapiens
CASSLGQGLANYGYT

SEQ ID NO: 2 - protein, 8 aa, Human Immunodeficiency Virus
FLKEKGGL

SEQ ID NO: 3 - protein, 12 aa, Homo sapiens
CAVRDLTGNQFY

SEQ ID NO: 4 - protein, 11 aa, Homo sapiens
CASSQGVTLLN

SEQ ID NO: 5 - protein, 9 aa, Human Immunodeficiency Virus
AIIRILQQL

SEQ ID NO: 6 - protein, 5 aa, Homo sapiens
CAETY

SEQ ID NO: 7 - protein, 14 aa, Homo sapiens
CASSIDGASNQPQH

SEQ ID NO: 8 - protein, 9 aa, Human Immunodeficiency Virus
SLYNTVATL

SEQ ID NO: 9 - protein, 16 aa, Homo sapiens
CAENSDAGGTSYGKLT

SEQ ID NO: 10 - protein, 15 aa, Homo sapiens
CATSRGAGSNTGELF

SEQ ID NO: 11 - protein, 12 aa, Homo sapiens
CAVRGSGTYKYI

SEQ ID NO: 12 - protein, 16 aa, Homo sapiens
CALSGNHSGGATNKLI

SEQ ID NO: 13 - protein, 10 aa, Human Immunodeficiency Virus
TSTLQEQIGW

SEQ ID NO: 14 - protein, 14 aa, Homo sapiens
CASSPWTGGGQPQH

SEQ ID NO: 15 - protein, 22 aa, Homo sapiens
CAASGGYQKVTFGTGTKLQVIP

SEQ ID NO: 16 - protein, 10 aa, Homo sapiens
CASTGTYGYT

SEQ ID NO: 17 - protein, 11 aa, Human Immunodeficiency Virus
KAFSPEVIPMF

SEQ ID NO: 18 - protein, 13 aa, Homo sapiens
CAMSAQQAGTALI

SEQ ID NO: 19 - protein, 9 aa, Human Immunodeficiency Virus
SEGATPQDL

SEQ ID NO: 20 - protein, 12 aa, Homo sapiens
CASSLVIMSEQY

SEQ ID NO: 21 - protein, 10 aa, Homo sapiens
CAATSGYALN

SEQ ID NO: 22 - protein, 8 aa, Human Immunodeficiency Virus
EIYKRWII

SEQ ID NO: 23 - protein, 14 aa, Homo sapiens
CASSVQGEFREKLF

SEQ ID NO: 24 - protein, 9 aa, Homo sapiens
CAGRDYKLS

SEQ ID NO: 25 - protein, 9 aa, Human Immunodeficiency Virus
KEKGGLEGL

SEQ ID NO: 26 - protein, 13 aa, Homo sapiens
CSARGDNPNTEAF

SEQ ID NO: 27 - protein, 11 aa, Homo sapiens
CAVNEYGQNFV

SEQ ID NO: 28 - protein, 9 aa, Hepatitis C Virus
ALYDVVTKL

SEQ ID NO: 29 - protein, 12 aa, Homo sapiens
CAWSGGLNTEAF

SEQ ID NO: 30 - protein, 11 aa, Homo sapiens
CASSVRTGELF

SEQ ID NO: 31 - protein, 10 aa, Human Immunodeficiency Virus
QVPLRPMTYK

SEQ ID NO: 32 - protein, 11 aa, Homo sapiens
CAASFTQNGLT

SEQ ID NO: 33 - protein, 15 aa, Homo sapiens
CASSERDSQYQETQY

SEQ ID NO: 34 - protein, 11 aa, Homo sapiens
CAASFTQNGLT

SEQ ID NO: 35 - protein, 11 aa, Homo sapiens
CASSPVLYEQY

SEQ ID NO: 36 - protein, 13 aa, Homo sapiens
CAVVAQGGSEKLV

SEQ ID NO: 37 - protein, 16 aa, Homo sapiens
CASSARAFPEGNQPQH

SEQ ID NO: 38 - protein, 13 aa, Homo sapiens
CAVVAQGGSEKLV

SEQ ID NO: 39 - protein, 13 aa, Homo sapiens
CASSETNRVMEAF

SEQ ID NO: 40 - protein, 11 aa, Homo sapiens
CAVSDPGFKTI

SEQ ID NO: 41 - protein, 15 aa, Homo sapiens
CATSAGRQRDTGELF

SEQ ID NO: 42 - protein, 11 aa, Homo sapiens
CAVSDPGFKTI

SEQ ID NO: 43 - protein, 10 aa, Homo sapiens
CASSNGYEQY

SEQ ID NO: 44 - protein, 10 aa, Homo sapiens
CAYRSDNDMR

SEQ ID NO: 45 - protein, 10 aa, Hepatitis C Virus
KLVALGINAV

SEQ ID NO: 46 - protein, 14 aa, Homo sapiens
CATSSQDGQVYEQY

SEQ ID NO: 47 - protein, 12 aa, Homo sapiens
CASYKAAGNKLT

SEQ ID NO: 48 - protein, 9 aa, Hepatitis C Virus
GPRLGVRAT

SEQ ID NO: 49 - DNA, 635 bp, Homo sapiens
  misc_feature (1)..(8): n is a, c, g, or t
  misc_feature (10)..(16): n is a, c, g, or t
  misc_feature (25)..(26): n is a, c, g, or t
  misc_feature (624)..(627): n is a, c, g, or t
  misc_feature (634)..(635): n is a, c, g, or t
nnnnnnnngnnnnnnntcgcccttnncagtggttcaacgcagagtacgcgggggggagacatcctctctagccccaactgtgccatgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtaatggatacgagcagtacttcgggccgggcaccaggctcacggtcacagaggacctgaaaaacgtgttcccacccgaggtcgctgtgtttgagccatcagaagcagagatctcccacaccaagggcgaattcgtttaaacctgcaggactagtccctttagtgagggttaattctgagcttggcgtaatcatggtcatagnnnntttcctnn

SEQ ID NO: 50 - DNA, 715 bp, Homo sapiens
  misc_feature (1)..(7): n is a, c, g, or t
  misc_feature (10)..(16): n is a, c, g, or t
  misc_feature (18)..(18): n is a, c, g, or t
  misc_feature (27)..(29): n is a, c, g, or t
  misc_feature (690)..(690): n is a, c, g, or t
  misc_feature (703)..(707): n is a, c, g, or t
  misc_feature (712)..(715): n is a, c, g, or t
nnnnnnnggnnnnnnnantcgcccttnnnagtggtatcaacgcagagtacgcggggagaagaggaggcttctcaccctgcagcagggacctgtgagcatggcatgccctggcttcctgtgggcacttgtgatctccacctgtcttgaatttagcatggctcagacagtcactcagtctcaaccagagatgtctgtgcagggggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcgacaatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtataagggcgaattcgtttaaacctgcaggactagtccctttagtgagggttaattctgagcttggcgtantcatggtcatagnnnnntttcnnnn

SEQ ID NO: 51 - DNA, 658 bp, Homo sapiens
  misc_feature (1)..(14): n is a, c, g, or t
  misc_feature (16)..(18): n is a, c, g, or t
  misc_feature (647)..(649): n is a, c, g, or t
  misc_feature (652)..(655): n is a, c, g, or t
  misc_feature (657)..(658): n is a, c, g, or t
nnnnnnnnnnnnnncnnntcgcccttaagcagtggtatcaacgcagagtacgcggggagagctggaaacacctccatcctgcctcttcatgccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagttcccaggacgggcaagtctacgagcagtacttcgggccgggcaccaggctcacggtcacagaggacctgaaaaacgtgttcccacccgaggtcgctgtgtttgagccatcagaagcagagatctcccacaccaagggcgaattcgtttaaacctgcaggactagtccctttagtgagggttaattctgagcttggcgtaatcatggtcatagctnnnttnnnngnn

SEQ ID NO: 52 - DNA, 715 bp, Homo sapiens
  misc_feature (1)..(13): n is a, c, g, or t
  misc_feature (15)..(16): n is a, c, g, or t
  misc_feature (18)..(18): n is a, c, g, or t
  misc_feature (31)..(31): n is a, c, g, or t
  misc_feature (700)..(700): n is a, c, g, or t
  misc_feature (703)..(705): n is a, c, g, or t
  misc_feature (707)..(708): n is a, c, g, or t
  misc_feature (710)..(715): n is a, c, g, or t
nnnnnnnnnnnnncnncnaattcgcccttangcagtggtatcaacgcagagtacgcgggggtttttctgctgtgggtacgtgagcaggaaacatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctcctacaaagctgcaggcaacaagctaacttttggaggaggaaccagggtgctagttaaaccaaatatccagaaccctgaccctgccgtgtaccagctgagagactctaaatccagtgacaagtctgtctgcctattcaccgattttgattctcaaacaaatgtgtcacaaagtaaggattctgatgtgtataagggcgaattcgtttaaacctgcaggactagtccctttagtgagggttaattctgagcttggcgtaatcatggtcntannntnntnnnnnn

SEQ ID NO: 53 - DNA, 812 bp, Homo sapiens
  misc_feature (1)..(10): n is a, c, g, or t
  misc_feature (12)..(15): n is a, c, g, or t
  misc_feature (17)..(19): n is a, c, g, or t
  misc_feature (22)..(22): n is a, c, g, or t
  misc_feature (32)..(32): n is a, c, g, or t
  misc_feature (722)..(722): n is a, c, g, or t
  misc_feature (744)..(746): n is a, c, g, or t
  misc_feature (783)..(783): n is a, c, g, or t
  misc_feature (796)..(801): n is a, c, g, or t
  misc_feature (810)..(810): n is a, c, g, or t
  misc_feature (812)..(812): n is a, c, g, or t
nnnnnnnnnngnnnncnnntcncccttatacncatcagaatccttactttgtgacacatttgtttgagaatcaaaatcggtgactaggcagacagacttgtcactggatttagagtctctcagctggtacacggcagggtcagggttctggatatagggcagcacggacaatctggttccgggaccaaagacaaaattctgaccatactcattcacggcacagaagtactcagctgtgtcactccactgcacagagggtttcctcagattaaaggagaatttactctttataaattcagcctcaaagcccttgatgcctttaaccagtggatcccctgaaaagtacttgaggagaagctgaaggtgttgaccagggtactggacataccagaagagattaacagttccaccgtaggaatagttgcatcccaactccagtgaggctgcttcagagagaattacgtggtggttatgctggctcacagactgggctctggcatctctcagggcaaaaatcatccccagcactggtatgagcaacaggagcatggctgagctgtggaaacactgcaagcgtctctttggaaattctcctcggggccagtaggaaagtggctggaacccaggtcttgagaatagcgagcgtgaggaaggttgggctaggcaagtctcttgttttggtaagaatccccgcgtactctgcgttgataccactgcttaanggcgaattcgtttaaacctgcnnnactagtccctttagtgagggttaattctgagcttggngtaatcatggtcnnnnnntgttttccngn

SEQ ID NO: 54 - DNA, 677 bp, Homo sapiens
  misc_feature (1)..(15): n is a, c, g, or t
  misc_feature (33)..(35): n is a, c, g, or t
  misc_feature (665)..(668): n is a, c, g, or t
  misc_feature (670)..(671): n is a, c, g, or t
  misc_feature (677)..(677): n is a, c, g, or t
nnnnnnnnnnnnnnntcgcccttggtgtgggannnctctgcttctgatggctcaaacacagcgacctcgggtgggaacaccttgttcaggtcctctacaactgtgagtctggtgccttgtccaaagaaagcttcagtgttcaggcctcccgaccaggcacagagatagaagccagagtcactgagaaggagcttcttagaactcaggatgaactgccggtcctggggtctggaggctgagagattctggggcacctcagagctgatctggccaataccaacggagtagaagagcagctggaggcccctgcctgcagcctgtcggtaccagtataggttggggtttgatgttccctccacagtgcactccagagagagcgggctgcccacaggctgcaccagggtcgctggccattgatgaatagtctgagatctgaccccaaagaaagtgcccaggagaagggcaaggagagagcagagcatcatccaagccagcctttccttcagctagtctgggggacagacgcctcctcttctgggccagtgatgtggccaggcacaccagtgtggcccgcgtactctgcgttgataccactgcttaagggcgaattcgcggccgctaaattcaattcgccctatagtgagtcgtattacaattcactggcnnnncnnttttan

SEQ ID NO: 55 - DNA, 695 bp, Homo sapiens
  misc_feature (1)..(13): n is a, c, g, or t
  misc_feature (683)..(683): n is a, c, g, or t
  misc_feature (685)..(685): n is a, c, g, or t
  misc_feature (688)..(688): n is a, c, g, or t
  misc_feature (695)..(695): n is a, c, g, or t
nnnnnnnnnnnnncgcccttatacacatcagaatccttactttgtgacacatttgtttgagaatcaaaatcggtgaataggcagacagacttgtcactggatttagagtctctcagctggtacacggcagggtcagggttctggatatggggtgtgaccaacagcgaggtgcctttgccgaagttgagtgcatacccggacgttgctgcacagaagtagacagccgagtcttcaggttgggtctctgtgatgtgcagggagaaatgtttggctgtcttgttcaatgtaacagcaattcgttggtctttcttttcgcccacatttgaacgaatgtctataataagctgaggtctttttccaagttcttgcttataccaagggaagtagtttgaggcactgtctgaataagtacacttgataacagcgctgtctccctcctggacactcagggttgaaggatgctgctccacattctctccattcaccaagtccagctgcagccacaggaatataaatacagctcgaatggatgtcatccttgttcttcccaattaagatcagtcattgacctgcaacctccagttatccccgcgtactctgcgttgataccactgcttaagggcgaattcgcggccgctaaattcaattcgccctatagtgagtcgtattacaattcactggcntnccntttttan

SEQ ID NO: 56 - DNA, 655 bp, Homo sapiens
  misc_feature (1)..(16): n is a, c, g, or t
  misc_feature (33)..(33): n is a, c, g, or t
  misc_feature (35)..(35): n is a, c, g, or t
  misc_feature (639)..(643): n is a, c, g, or t
  misc_feature (652)..(655): n is a, c, g, or t
nnnnnnnnnnnnnnnncgcccttggtgtgggananctctgcttctgatggctcaaacacagcgacctcgggtgggaacaccttgttcaggtcctccaagacagagagctgggttccactgccaaaaaacagtttttctctaaactccccctgtacgctgctggcacagaaatacaaagctgagtcccccagctccagagagctcaggtttagttcagagtgcaagtcagggaactgttgtgcggagaatcgttcaagaatgtttccttttgctctctcttctccattataatactgaatgaggaactggaggccctggtccaggctctgttggtaccagtacacagagaggtctccagacctaggggagcatctcagcgtcactcgctgtccagttgctgtgatcaggtgctttggggtttgtgtgactccagaatccactgggcctgctcccaggagacaaaaggccacacagcagaggagcctgaagcccatggcaggatctcctagcttggggctggtgtctctgtagtaagcattctccccgcgtactctgcgttgataccactgcttaagggcgaattcgcggccgctaaattcaattcgccctatagtgagtcgtattacaattcactggccnnnnntttttacannnn

SEQ ID NO: 57 - DNA, 765 bp, Homo sapiens
  misc_feature (1)..(13): n is a, c, g, or t
  misc_feature (599)..(599): n is a, c, g, or t
  misc_feature (609)..(610): n is a, c, g, or t
  misc_feature (614)..(614): n is a, c, g, or t
  misc_feature (617)..(617): n is a, c, g, or t
  misc_feature (619)..(619): n is a, c, g, or t
  misc_feature (650)..(650): n is a, c, g, or t
  misc_feature (687)..(687): n is a, c, g, or t
  misc_feature (695)..(696): n is a, c, g, or t
  misc_feature (699)..(699): n is a, c, g, or t
  misc_feature (702)..(703): n is a, c, g, or t
  misc_feature (705)..(706): n is a, c, g, or t
  misc_feature (737)..(737): n is a, c, g, or t
  misc_feature (752)..(758): n is a, c, g, or t
  misc_feature (764)..(765): n is a, c, g, or t
nnnnnnnnnnnnncgcccttatacacatcagaatccttactttgtgacacatttgtttgagaatcaaaatcggtgaataggcagacagacttgtcactggatttagagtctctcagctggtacacggcagggtcagggttctggatatttgctcttacagttactgtggttccggctccaaagctgagcttgtagtcgcgtcctgcacagaggtagaggcctgtatcaccaggctgggccgcagtgatgtggagagaactgtcctttcttgcatcaccaaactgaaaggttagtctcttcagcttcttcacttctccacccgtaactactgtcaccaggaggacaggaccttccccaggctcctgtctgtaccattgtaagctggaaaaaacacttgaggagttgcagtacacagtgagattttctccctcttggatgcttagaaactgagggctctgctccagcagctgggtgctcacccatgccaactgaatccaaagaatggacacggagaatttcaggaccatcttgtctttctatcacatggtggacatggcccctgactttagctgctcctgaaagagcccgtcctggaacanacttctctgnnctanaanantgcttgctgccacccactttgagttccatanaaagccccccgcgactctgcgttgataccactgcttnagggcganntcncgnncnntaaattcaattcgccctatagtgagtcgtantacaattcactggcnnnnnnnttttann

SEQ ID NO: 58 - DNA, 670 bp, Homo sapiens
  misc_feature (1)..(7): n is a, c, g, or t
  misc_feature (10)..(12): n is a, c, g, or t
  misc_feature (14)..(15): n is a, c, g, or t
  misc_feature (17)..(17): n is a, c, g, or t
  misc_feature (27)..(27): n is a, c, g, or t
  misc_feature (657)..(662): n is a, c, g, or t
  misc_feature (669)..(670): n is a, c, g, or t
nnnnnnnggnnncnnantcgcccttangcagtggtatcaacgcagagtacgcggtaagcagtggtatcaacgcagagtacgcgggagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagaggggacaacccgaacactgaagctttctttggacaaggcaccagactcacagttgtagaggacctgaacaaggtgttcccacccgaggtcgctgtgtttgagccatcagaagcagagatctcccacaccaagggcgaattcgtttaaacctgcaggactagtccctttagtgagggttaattctgagcttggcgtaatcatggtcatannnnnntttcctnn

SEQ ID NO: 59 - DNA, 678 bp, Homo sapiens
  misc_feature (1)..(12): n is a, c, g, or t
  misc_feature (663)..(670): n is a, c, g, or t
  misc_feature (674)..(678): n is a, c, g, or t
nnnnnnnnnnnncgcccttatacacatcagaatccttactttgtgacacatttgtttgagaatcaaaatcggtgaataggcagacagacttgtcactggatttagagtctctcagctggtacacggcagggtcaggcttctggatatagggaataacggtgagtctcgttccagtcccaaagaagaggttgtttgccccagtttgataagtctctgcacagaagtagatagctgagtccccagtctgggtgtctgcaatgcgcagagacagatgtttatcctttttattcaatagaacagtgagtctttggtcttgtttcatgtccatatttgagaaaatatacgtcagcaactggagacctgctccagattcttgcttataccagtataagtaggtggaggagctgtctgtgtaagtgcagtttataacggagctgtctccctctcggacactcaggaaaagactctgctccacatcctctcctctactcatacagtccagctgcagccacaaaaacaggaacgaaaatccagcaaatgtcttcattgttctccccactgggacctgccccgcgtactctgcgttgataccactgcttaagggcgaattcgcggccgctaaattcaattcgccctatagtgagtcgtattacaattcactgnnnnnnnntttnnnnn

SEQ ID NO: 60 - DNA, 650 bp, Homo sapiens
  misc_feature (1)..(15): n is a, c, g, or t
  misc_feature (34)..(34): n is a, c, g, or t
  misc_feature (650)..(650): n is a, c, g, or t
nnnnnnnnnnnnnnncgcccttggtgtgggaganctctgcttctgatggctcaaacacagcgacctcgggtgggaacacgtttttcaggtcctctagcacggtgagccgtgtccctggcccgaagaactgctcattcaacaaagtcaccccttggctgctggcacagaaataaactccagaatcctccagttctgcaggctgcaccttcagagtagaatacgtccctccagtcctttcagctaagaatcgattgttgggcataccggactcatcctgtttagactctttcacaaaatgtaacagaaattttatttcttttcccataacacgtcgataccaataaagattatcatgtccagaaattgggtcacatctcagagtcacagtctggcccttctctattacgctgtggctggggaactgagtaactccagcttctatgtgctaagcatgagaaaaaggaaagcaaatctgtctcttggccctgtaagatgtggcctccagtgacatcagtatattagccaatgtccacagtctcaggagctccctctaccccgcgtactctgcgttgataccactgcttaagggcgaattcgcggccgctaaattcaattcgccctatagtgagtcgtattacaattcactggcn

SEQ ID NO: 61 - DNA, 678 bp, Homo sapiens
  misc_feature (1)..(4): n is a, c, g, or t
  misc_feature (6)..(7): n is a, c, g, or t
  misc_feature (9)..(10): n is a, c, g, or t
  misc_feature (12)..(12): n is a, c, g, or t
  misc_feature (21)..(21): n is a, c, g, or t
  misc_feature (462)..(462): n is a, c, g, or t
  misc_feature (586)..(586): n is a, c, g, or t
  misc_feature (641)..(641): n is a, c, g, or t
  misc_feature (651)..(651): n is a, c, g, or t
  misc_feature (658)..(669): n is a, c, g, or t
  misc_feature (672)..(678): n is a, c, g, or t
nnnngnncnnantcgcccttnagcagtggtatcaacgcagagtacgcggggaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgagtttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgcgaggctcaggaacctacaaatacatctttggaacaggcaccaggctgaaggttttagcaaatatccngaaccctgaccctgccgtgtaccagctgagagactctaaatccagtgacaagtctgtctgcctattcaccgattttgattctcaaacaaatgtgtcacaaagtaaggattctgatgtgtataanggcgaattcgtttaaacctgcaggactagtccctttagtgagggttaattctgancttggcgtantcatggnnnnnnnnnnnnttnnnnnnn

SEQ ID NO: 62 - DNA, 663 bp, Homo sapiens
  misc_feature (1)..(8): n is a, c, g, or t
  misc_feature (10)..(13): n is a, c, g, or t
  misc_feature (23)..(23): n is a, c, g, or t
  misc_feature (637)..(637): n is a, c, g, or t
  misc_feature (644)..(652): n is a, c, g, or t
  misc_feature (654)..(654): n is a, c, g, or t
  misc_feature (658)..(658): n is a, c, g, or t
  misc_feature (660)..(661): n is a, c, g, or t
  misc_feature (663)..(663): n is a, c, g, or t
nnnnnnnncnnnntcgcccttangcagtggtatcacgcagagtcgcgggggagacagacagatgcttcattcctgcatggggtggtattcctgccatgggtcctgggcttctccactggatggccctttgtctccttggaacaggtcacggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagaggggcaggatcgaacaccggggagctgttttttggagaaggctctaggctgaccgtactggaggacctgaaaaacgtgttcccacccgaggtcgctgtgtttgagccatcagaagcagagatctcccacaccaagggcgaattcgtttaaacctgcaggactagtccctttagtgagggttaattctgagcttggcgtantcatggnnnnnnnnntntttncnngn

SEQ ID NO: 63 - DNA, 721 bp, Homo sapiens
  misc_feature (1)..(7): n is a, c, g, or t
  misc_feature (9)..(11): n is a, c, g, or t
  misc_feature (13)..(14): n is a, c, g, or t
  misc_feature (16)..(16): n is a, c, g, or t
  misc_feature (24)..(25): n is a, c, g, or t
  misc_feature (256)..(256): n is a, c, g, or t
  misc_feature (513)..(513): n is a, c, g, or t
  misc_feature (716)..(721): n is a, c, g, or t
nnnnnnngnnncnnantcgccctnnagcagtggtatcaacgcagagtacgcggggatggctggagattgcaggtttatgactgatcctatttgggaagaacaatgatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcanactacttcatttggtacaagcaagaatctggaaaagatcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaattctgatgctggtggtactagctatggaaagctgacatttggacaagggaccatcttgactgtccatccnaatatccagaagcctgaccctgccgtgtaccagctgagagactctaaatccagtgacaagtctgtctgcctattcaccgattttgattctcaaacaaatgtgtcacaaagtaaggattctgatgtgtataagggcgaattcgtttaaacctgcaggactagtccctttagtgagggttaattctgagcttggcgtatcatgtnnnnnn

SEQ ID NO: 64 - DNA, 578 bp, Homo sapiens
  misc_feature (1)..(8): n is a, c, g, or t
  misc_feature (10)..(13): n is a, c, g, or t
  misc_feature (15)..(15): n is a, c, g, or t
  misc_feature (17)..(17): n is a, c, g, or t
  misc_feature (567)..(571): n is a, c, g, or t
  misc_feature (574)..(578): n is a, c, g, or t
nnnnnnnngnnnncnanttcgcccttccctttgcactatgagcaacatttgtttcctgggagcaaacaccgtggatggtggaatcactcagtccccgaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggaggtatagctgaagggtacagcgtctctcgggagaagaaggaatcctttcctctcactgtgacatcggcccaaaagaacccgacagctttctatctctgtgccagtagtatagatggcgctagcaatcagccccagcattttggtgatgggactcgactctccatcctagaggacctgaacaaggtgttcccacccgaggtcgctgtgtttgagccatcagaagcagagatctcccacaccaagggcgaattcgtttaaacctgcaggactagtccctttagtgagggttaattctgagcttggcgtaatcatggtcatannnnnttnnnnn

SEQ ID NO: 65 - DNA, 550 bp, Homo sapiens
  misc_feature (1)..(12): n is a, c, g, or t
  misc_feature (14)..(19): n is a, c, g, or t
  misc_feature (29)..(29): n is a, c, g, or t
  misc_feature (526)..(528): n is a, c, g, or t
  misc_feature (530)..(530): n is a, c, g, or t
  misc_feature (540)..(540): n is a, c, g, or t
  misc_feature (543)..(544): n is a, c, g, or t
  misc_feature (548)..(550): n is a, c, g, or t
nnnnnnnnnnnntnnnnnngtcctctacnacggttaacctggtccccgaaccgaaggtgtagccatagttagctaagccctgccctaaactgctggcacagaagtacggagaggtctggttggggctgggcgactccaggatcagggggaaattcctcttctcttttcgagagactttgtacccttcaggaacacctcccttatcagtcacctcaacattcattgaatagtagatctgccttaagcccagccctgggtcttgtcgataccaggacatatactcatggttcatattctgagaacaagtcactgttaacttctttccagtcactgtgatgaggtatcttgggttctgggtcacttgggcttccagggggcctgctcctagaaggcaaaggaccacatagccaaggagctgggggcccatggcagcatcaggcaggtgtctgccagttctgggggctccaggtggtttctgtaacgtctccacctcttcccccgcgtactctgcgttgataccactgcnnncnctgcgttganacnnctgnnn

SEQ ID NO: 66 - DNA, 550 bp, Homo sapiens
  misc_feature (9)..(14): n is a, c, g, or t
  misc_feature (24)..(24): n is a, c, g, or t
  misc_feature (32)..(33): n is a, c, g, or t
  misc_feature (328)..(328): n is a, c, g, or t
  misc_feature (474)..(474): n is a, c, g, or t
  misc_feature (546)..(550): n is a, c, g, or t
gaccccccnnnnnncgcccgccgngagcttanntggagccatggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaanaaaccatctgcccttgtgagcgactccgctttgtacttctgtgctgtgagagacctcaccggtaaccagttctattttgggacagggacaagtttgacggtcattccaaatatccagaaccctgaccctgccgtgtaccagctganagactctaaatccagtgacaagtctgtctgcctattcaccgattttgattctcaaacaaatgtgtcacaaannnnn

SEQ ID NO: 67 - DNA, 791 bp, Homo sapiens
  misc_feature (1)..(3): n is a, c, g, or t
  misc_feature (6)..(6): n is a, c, g, or t
  misc_feature (10)..(11): n is a, c, g, or t
  misc_feature (697)..(697): n is a, c, g, or t
  misc_feature (720)..(720): n is a, c, g, or t
  misc_feature (743)..(743): n is a, c, g, or t
  misc_feature (753)..(753): n is a, c, g, or t
  misc_feature (756)..(756): n is a, c, g, or t
  misc_feature (758)..(758): n is a, c, g, or t
  misc_feature (761)..(762): n is a, c, g, or t
  misc_feature (766)..(782): n is a, c, g, or t
  misc_feature (784)..(791): n is a, c, g, or t
nnnggncgcnnattcgcccttaagcagtggtatcaacgcagagtacgcggggcagtaactttgctagtacctcttgagtgcaaggtggagaattaagatctggatttgagacggagcacggaacatttcactcaggggaagagctatgaacatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtggaaatcactcaggtggtgctacaaacaagctcatctttggaactggcactctgcttgctgtccggccaaatatccagaaccctgaccctgccgtgtaccagctgagagactctaaatccagtgacaagtctgtctgcctattcaccgattttgattctcaaacaaatgtgtcacaaagtaaggattctgatgtgtataanggcgaattcgtttaaacctgcanggactagtccctttagtgagggntaattctganctngncgnnatcnnnnnnnnnnnnnnnnntnnnnnnnn

SEQ ID NO: 68 - DNA, 629 bp, Homo sapiens
  misc_feature (1)..(7): n is a, c, g, or t
  misc_feature (9)..(12): n is a, c, g, or t
  misc_feature (14)..(16): n is a, c, g, or t
  misc_feature (27)..(27): n is a, c, g, or t
  misc_feature (613)..(613): n is a, c, g, or t
  misc_feature (617)..(620): n is a, c, g, or t
  misc_feature (627)..(627): n is a, c, g, or t
nnnnnnngnnnncnnnttcgcccttangcagtgtatcaacgcagagtacgcgggaagcagtggtatcaacgcagagtacgcgggaagcagtggtatcaacgcagagtacgcgggaagcagtggtatcaacgcagagtacgcgggaagcagtggtatcaacgcagagtacgcgggaagcagtggtatcaacgcagagtacgcgggggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagcccgtggacagggggcggccagccccagcattttggtgatgggactcgactctccatcctagaggacctgaacaaggtgttcccacccgaggtcgctgtggttgagccatcagaagcgagatctcccacaccaagggcgaattcgtttaaacctgcaggactagtccctttagtgagggttaattctgagcttggcgtaatcatggtcntagnnnngtttccnga

SEQ ID NO: 69 - DNA, 822 bp, Homo sapiens
  misc_feature (1)..(14): n is a, c, g, or t
  misc_feature (17)..(17): n is a, c, g, or t
  misc_feature (23)..(25): n is a, c, g, or t
  misc_feature (27)..(27): n is a, c, g, or t
  misc_feature (29)..(29): n is a, c, g, or t
  misc_feature (33)..(34): n is a, c, g, or t
  misc_feature (36)..(37): n is a, c, g, or t
  misc_feature (41)..(42): n is a, c, g, or t
  misc_feature (45)..(45): n is a, c, g, or t
  misc_feature (47)..(48): n is a, c, g, or t
  misc_feature (59)..(59): n is a, c, g, or t
  misc_feature (67)..(70): n is a, c, g, or t
  misc_feature (74)..(74): n is a, c, g, or t
  misc_feature (723)..(725): n is a, c, g, or t
  misc_feature (727)..(727): n is a, c, g, or t
  misc_feature (747)..(749): n is a, c, g, or t
  misc_feature (763)..(763): n is a, c, g, or t
  misc_feature (780)..(780): n is a, c, g, or t
  misc_feature (785)..(786): n is a, c, g, or t
  misc_feature (793)..(793): n is a, c, g, or t
  misc_feature (797)..(802): n is a, c, g, or t
  misc_feature (804)..(809): n is a, c, g, or t
  misc_feature (813)..(813): n is a, c, g, or t
  misc_feature (816)..(816): n is a, c, g, or t
  misc_feature (818)..(822): n is a, c, g, or t
nnnnnnnnnnnnnntcncccttnnncngnggtnncnncgcnnagnanncgggggaaganatacttgnnnntatngctctcttggctggagattgcaggtcccagtggggagaacaatgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagcttctgggggttaccagaaagttacctttggaactggaacaaagctccaagtcatcccaaatatccagaagcctgaccctgccgtgtaccagctgagagactctaaatccagtgacaagtctgtctgcctattcaccgattttgattctcaaacaaatgtgtcacaaagtaaggattctgatgtgtatatcacagacaaaactgtccatagacctcatgtctagcacagttttgtctgtgatcccgcgtactctgcgttgataccactgcttannngncgaattcgtttaaacctgcnnnactagtccctttantgagggttaattctgancttgnngtaatcntggnnnnnncnnnnnntttnccngnnnnn

SEQ ID NO: 70 - DNA, 700 bp, Homo sapiens
  misc_feature (1)..(9): n is a, c, g, or t
  misc_feature (11)..(11): n is a, c, g, or t
  misc_feature (13)..(14): n is a, c, g, or t
  misc_feature (16)..(16): n is a, c, g, or t
  misc_feature (673)..(673): n is a, c, g, or t
  misc_feature (682)..(687): n is a, c, g, or t
  misc_feature (689)..(691): n is a, c, g, or t
  misc_feature (694)..(694): n is a, c, g, or t
  misc_feature (697)..(697): n is a, c, g, or t
  misc_feature (699)..(700): n is a, c, g, or t
nnnnnnnnncncnnantcgcccttaagcagtggtatcaacgcagagtacgcggggacattaggccaggagaagcccccgagccaagtctcttttctcattctcttccaacaagtgcttggagctccaagaaggccccctttgcactatgagcaaccaggtgctctgctgtgtggtcctttgtctcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagatcgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtaccgggacttatggctacaccttcggttcggggaccaggttaaccgttgtagaggacctgaacaaggtgttcccacccgaggtcgctgtgtttgagccatcagaagcagagatctcccacaccaagggcgaattcgtttaaacctgcaggactagtccctttagtgagggttaattctgagcttggcgtantcatggtcnnnnnntnnnttnccngnn

SEQ ID NO: 71 - DNA, 790 bp, Homo sapiens
  misc_feature (1)..(7): n is a, c, g, or t
  misc_feature (9)..(12): n is a, c, g, or t
  misc_feature (14)..(16): n is a, c, g, or t
  misc_feature (25)..(25): n is a, c, g, or t
  misc_feature (652)..(652): n is a, c, g, or t
  misc_feature (697)..(697): n is a, c, g, or t
  misc_feature (760)..(761): n is a, c, g, or t
  misc_feature (765)..(770): n is a, c, g, or t
  misc_feature (773)..(775): n is a, c, g, or t
  misc_feature (777)..(777): n is a, c, g, or t
  misc_feature (782)..(790): n is a, c, g, or t
nnnnnnngnnnncnnntcgcccttnagcagtggtatcaacgcagagtacgcggggaggacagatttcttttatgattcctacagcagaaaaatgagaaacgtttgttattatttttttttcgtgtttaaagtttgaatcctcagtgaaccagggcagaaaagaatgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagcgcgcaacaggcaggaactgctctgatctttgggaagggaaccaccttatcagtgagttccaatatccagaaccctgaccctgccgtgtaccagctgagagactctaaatccagtgacaagtctgtctgcctattcaccgattttgantctcaaacaaatgtgtcacaaagtaaggattctgatgtgtataanggcgaattcgtttaaacctgcaggactagtccctttagtgagggttaattctgagcttggcgnnatcnnnnnnaannntnttttnnnnnnnnn

SEQ ID NO: 72 - DNA, 674 bp, Homo sapiens
  misc_feature (1)..(8): n is a, c, g, or t
  misc_feature (10)..(12): n is a, c, g, or t
  misc_feature (14)..(16): n is a, c, g, or t
  misc_feature (36)..(36): n is a, c, g, or t
  misc_feature (647)..(648): n is a, c, g, or t
  misc_feature (656)..(664): n is a, c, g, or t
  misc_feature (668)..(668): n is a, c, g, or t
  misc_feature (671)..(674): n is a, c, g, or t
nnnnnnnngnnncnnntcgcccttggtgtgggaganctctgcttctgatggctcaaacacagcgacctcgggtgggaacacgtttttcaggtcctctgtgaccgtgagcctggtgcccggcccgaagtactgctcgctcatggtgactaagctgctggcacagagatacacggccgagtcctcaagctttgcaggctggatcttgagagtggagtctactcctttgagcctctctgcagaaaatcgatccttaggcaactgtgaatcatccactacaccgttattctgaaactgaatcagaagctttgggccctgtcccaggatctgctggtaccagtaaagggtagcatggccagatataggattgcaccaaaaagccacactctgccttttctctataatcttatatctgggagactgggcaactccagcttctgtgagttctgctcccaggagacagagggccgcccagcagaggagcctggtgcccatggcagggtcagggaaggatgggagctttgcccaatcaaggtcactgtgagcaacagcccccgcgtactctgcgttgataccactgcttaagggcgaattcgtttaaacctgcaggactagtccctttagtgagggttaattctgagcttggcgtnntcatggtnnnnnnnnntttnccnnnn

SEQ ID NO: 73 - DNA, 419 bp, Homo sapiens
  misc_feature (1)..(11): n is a, c, g, or t
  misc_feature (13)..(13): n is a, c, g, or t
  misc_feature (33)..(33): n is a, c, g, or t
  misc_feature (319)..(319): n is a, c, g, or t
  misc_feature (322)..(323): n is a, c, g, or t
  misc_feature (409)..(411): n is a, c, g, or t
  misc_feature (417)..(419): n is a, c, g, or t
nnnnnnnnnnnantcgcccttggtgtgggaganctctgcttctgatggctcaaacacagcgacctcgggtgggaacacgtttttcaggtcctccagtacggtcagcctagagccttctccaaaaaacagctccccggtccgtacgctgctggcacagaagtacagagaggtctggttggggctcggcgactccaggatcagggggaaattcctcttctcttttcgagagactttgtacccttcaggaacatctcccttatcagtcacctcaacattcattgaatagtagatctgccttaagcccagccctgggtcttgntgnnaccagaacagaattcgagaagggcgaattcgcggccgctaaattcaattcgccctatagtgagtcgtattacaattcactggccgnnnttttannn

SEQ ID NO: 74 - DNA, 432 bp, Homo sapiens
  misc_feature (1)..(13): n is a, c, g, or t
  misc_feature (33)..(33): n is a, c, g, or t
  misc_feature (340)..(341): n is a, c, g, or t
  misc_feature (421)..(422): n is a, c, g, or t
  misc_feature (430)..(430): n is a, c, g, or t
  misc_feature (432)..(432): n is a, c, g, or t
nnnnnnnnnnnnntcgcccttggtgtgggaganctctgcttctgatggctcaaacacagcgacctcgggtgggaacacgtttttcaggtcctcgagcaccaggagccgcgtgcctggcccgaagtactgggtctcttggtactgactgtccctctcgctgctggcacagaaatacaaagctgagtcccccagctccagagagctcaggtttagttcagagtgcaagtcagggaactgttgtgcggagaatcgttcaagaatgtttccttttgctctctcttctccattataatactgaatgaggaactggaggccctggtccaggctctgatggtaccannacagaattcgagaagggcgaattcgcggccgctaaattcaattcgccctatagtgagtcgtattacaattcactggccgnncgttttanan

SEQ ID NO: 75 - DNA, 755 bp, Homo sapiens
  misc_feature (1)..(12): n is a, c, g, or t
  misc_feature (14)..(14): n is a, c, g, or t
  misc_feature (24)..(24): n is a, c, g, or t
  misc_feature (680)..(680): n is a, c, g, or t
  misc_feature (682)..(682): n is a, c, g, or t
  misc_feature (741)..(745): n is a, c, g, or t
  misc_feature (747)..(750): n is a, c, g, or t
  misc_feature (754)..(755): n is a, c, g, or t
nnnnnnnnnnnnantcgcccttancagtggtatcaacgcagagtacgcggggggacatgaataaagcacaggaggttgaagtcagatttgcagctttctaggcgggagacaagacaatctgcatcttcacaggggggatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattgaggataaaaatgaagatggaagattcactgtcttcttaaacaaaagtgccaagcacctctctctgcacattgtgccctcccagcctggagactctgcagtgtacttctgtgcagcaagcttcacgcagaacggactcacctttggcaaagggactcatctaatcatccagccctatatccagaaccctgaccctgccgtgtaccagctgagagactctaactccagtgacaagtctgtctgcctattcaccgattttgattctcaaacaaatgtgtcacaaagtaaggattctgatgtgtataangncgaattcgcggccgctaaattcaattcgccctatagtgagtcgtattacaattcactgnnnnncnnnntttnn

SEQ ID NO: 76 - DNA, 421 bp, Homo sapiens
  misc_feature (1)..(13): n is a, c, g, or t
  misc_feature (30)..(30): n is a, c, g, or t
  misc_feature (32)..(32): n is a, c, g, or t
  misc_feature (411)..(411): n is a, c, g, or t
  misc_feature (418)..(421): n is a, c, g, or t
nnnnnnnnnnnnncgcccttggtgtgggananctctgcttctgatggctcaaacacagcgacctcgggtgggaacacgtttttcaggtcctctgtgaccgtgagcctggtgcccggcccgaagtactgctcgtatagaacggggctgctggcacagaaataaactccagaatcctccagttctgcaggctgcaccttcagagtagaatacgtccctccagtcctttcagctaagaatcgattgttgggcataccggactcatcctgtttagactctttcacaaaatgtaacagaaattttatttcttttcccataacatgtcgataccagtacagaattcgagaagggcgaattcgcggccgctaaattcaattcgccctatagtgagtcgtattacaattcactggccgncgttttnnnn

SEQ ID NO: 77 - DNA, 436 bp, Homo sapiens
  misc_feature (1)..(5): n is a, c, g, or t
  misc_feature (7)..(13): n is a, c, g, or t
  misc_feature (33)..(33): n is a, c, g, or t
  misc_feature (35)..(35): n is a, c, g, or t
  misc_feature (48)..(48): n is a, c, g, or t
  misc_feature (434)..(434): n is a, c, g, or t
  misc_feature (436)..(436): n is a, c, g, or t
nnnnntnnnnnnnattcgcccttggtgtgggananctctgcttctganggctcaaacacagcgacctcgggtgggaacaccttgttcaggtcctctaggatggagagtcgagtcccatcaccaaaatgctggggctgattgccctctgggaaggcccgggcgctgctggcacagaaatacaaagctgagtcccccagctccagagagctcaggtttagttcagagtgcaagtcagggaactgttgtgcggagaatcgttcaagaatgtttccttttgctctctcttctccattataatagtgaatgaggaactggaggccctggtccaggctctgacggtaccagtacagaattcgagaagggcgaattcgcggccgctaaattcaattcgccctatagtgagtcgtattacaattcactggccgtcgttttanan

SEQ ID NO: 78 - DNA, 436 bp, Homo sapiens; identical to SEQ ID NO: 77
  misc_feature (1)..(5): n is a, c, g, or t
  misc_feature (7)..(13): n is a, c, g, or t
  misc_feature (33)..(33): n is a, c, g, or t
  misc_feature (35)..(35): n is a, c, g, or t
  misc_feature (48)..(48): n is a, c, g, or t
  misc_feature (434)..(434): n is a, c, g, or t
  misc_feature (436)..(436): n is a, c, g, or t

SEQ ID NO: 79 - DNA, 425 bp, Homo sapiens
  misc_feature (1)..(8): n is a, c, g, or t
  misc_feature (10)..(13): n is a, c, g, or t
  misc_feature (33)..(34): n is a, c, g, or t
  misc_feature (425)..(425): n is a, c, g, or t
nnnnnnnnannnnttcgcccttggtgtgggagnnctctgcttctgatggctcaaacacagcgacctcgggtgggaacaccttgttcaggtcctctacaactgtgagtctggtgccttgtccaaagaaagcttccattaccctgtttgtttcactgctggcgcagaaatacacagatgtctgggagcgggtagctgactccagagtgagggggaaattctctgtcttggatctggagacaacatagccatcggggacttctcctttatctgtaatatcagcagctgctgagtaatagatcagcctcagcccatgtcccaggtcttgacggtaccagaacagaattcgagaagggcgaattcgcggccgctaaattcaattcgccctatagtgagtcgtattacaattcactggccgtcgttttacn

SEQ ID NO: 80 - DNA, 430 bp, Homo sapiens
  misc_feature (1)..(12): n is a, c, g, or t
  misc_feature (30)..(32): n is a, c, g, or t
  misc_feature (428)..(430): n is a, c, g, or t
nnnnnnnnnnnntcgcccttggtgtgggannnctctgcttctgatggctcaaacacagcgacctcgggtgggaacacgtttttcaggtcctccagtacggtcagcctagagccttctccaaaaaacagctccccggtgtctcgctgcctcccggcactggtggcacagaagtaaagagctgtctggttggggatggcagactctagggacagggagaatttagcctgtgcctgtcgagagacactgtatccatcagagatctctcctttgtttatatctttgacatcaaaggagtaatagatcaactgtaggcccagtcctgggtcttgatggtaccaatacagaattcgagaagggcgaattcgcggccgctaaattcaattcgccctatagtgagtcgtattacaattcactggccgtcgttttannn

SEQ ID NO: 81 - DNA, 788 bp, Homo sapiens
  misc_feature (1)..(13): n is a, c, g, or t
  misc_feature (15)..(17): n is a, c, g, or t
  misc_feature (30)..(30): n is a, c, g, or t
  misc_feature (769)..(770): n is a, c, g, or t
  misc_feature (787)..(788): n is a, c, g, or t
nnnnnnnnnnnnnannntcgcccttatacncatcagaatccttactttgtgacacatttgtttgagaatcaaaatcggtgaataggcagacagacttgtcactggatttagagtctctcagctggtacacggcagggtcagggttctggatatttgctttaacaaatagtcttgttcctgctccaaagatagttttgaagcctggatcactcacagcacagaagtactcagccgtgtcgcttatatggactgagggtttcctcaagtggaaggaagtttgactcttgttaaattcagcctcaaaaccgttgatgcctttaaccagggtggatcctgataaatacttcaggagaagctggagtccttggttggggtattgcacataccagaagagatacactgaaacagacgatgagtagttgcacctcagcaccacaggggcttcttcaaagacagggacttggctgtcaagctgggtcacaggctgggctctggttcctccccgggtaaaaatcacctggaacgctgggacgagcagcaggagcatggctgagcagtggcaatgctgcaggaccttgagctgggcggacagaagccaagggcgctgagcctcaggagctaggaactgtgaggaggttggattggacaagtccctggctttgaaaagtttcagaaacagccccgcgtactccccgcgtactctgcgttgataccactgcttaagggcgaattcgcggccgctaaattcaattcgccctatagtgagtcnnattacaattcactggcnn

SEQ ID NO: 82 - protein, 15 aa, Homo sapiens
CASSSPKDPSNQPQH

SEQ ID NO: 83 - protein, 10 aa, Human Immunodeficiency Virus
KRWIILGLNK

SEQ ID NO: 84 - protein, 14 aa, Homo sapiens
CAEDPTSSSGYALN

SEQ ID NO: 85 - DNA, 702 bp, Homo sapiens
  misc_feature (1)..(8): n is a, c, g, or t
  misc_feature (11)..(13): n is a, c, g, or t
  misc_feature (15)..(17): n is a, c, g, or t
  misc_feature (679)..(679): n is a, c, g, or t
  misc_feature (681)..(691): n is a, c, g, or t
  misc_feature (696)..(702): n is a, c, g, or t
nnnnnnnnggnnncnnntcgcccttaagcagtggtatcaacgcagagtacgcggggcaggtcccagtggggagaacaatgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatggaacaagaccaaagactcactgttctattgaataaaaaggataaacatctgtctctgcgcattgcagacacccagactggggactcagctatctacttctgtgcagaggatcccacctcaagttccgggtatgcacccaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtataagggcgaattcgtttaaacctgcaggactagtccctttagtgagggttaattctgagcttggcgtaatcntnnnnnnnnnnnttttnnnnnnn

SEQ ID NO: 86 - DNA, 664 bp, Homo sapiens
  misc_feature (1)..(12): n is a, c, g, or t
  misc_feature (15)..(17): n is a, c, g, or t
  misc_feature (26)..(26): n is a, c, g, or t
  misc_feature (640)..(651): n is a, c, g, or t
  misc_feature (657)..(664): n is a, c, g, or t
nnnnnnnnnnnngcnnntcgcccttnagcagtggtatcaacgcagagtacgcgggggatctggtaaagctcccatcctgccctgaccctgccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcagtcccaaagatcctagcaatcagccccagcattttggtgatgggactcgactctccatcctagaggacctgaacaaggtgttcccacccgaggtcgctgtgtttgagccatcagaagcagagatctcccacaccaagggcgaattcgtttaaacctgcaggactagtccctttagtgagggttaattctgagcttggcgtaatcnnnnnnnnnnnntttttnnnnnnnn